SEQUENCE LISTING (32 sequences)

SEQ ID NO: 1 - DNA, 158 bp, Homo sapiens
cagcttgggcacgaagacctacacctgcaacgtagatcacaagcccagcaacaccaaggtggacaagagagttgagtccaaatatggtcccccatgcccatcatgcccagcacctgagttcctgggggaccatcagtcttcctgttccccccaaaacc

SEQ ID NO: 2 - DNA, 981 bp, Homo sapiens
gcaagcttcaagggcccatcggtcttccccctggtgccctgctccaggagcacctccgagagcacagccgccctgggctgcctggtcaaggactacttccccgaaccggtgacggtgtcgtggaactcatgcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagggtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggaggacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 3 - DNA, 17 bp, Artificial Sequence
  synthetically generated oligonucleotide probe
accaggcagcccagggc

SEQ ID NO: 4 - DNA, 21 bp, Artificial Sequence
  synthetically generated oligonucleotide probe
ggttcggggaagtagtccttg

SEQ ID NO: 5 - DNA, 21 bp, Artificial Sequence
  synthetically generated oligonucleotide probe
gagttccacgacaccgtcacc

SEQ ID NO: 6 - DNA, 18 bp, Artificial Sequence
  synthetically generated oligonucleotide probe
ccgctggtcagggcgcat

SEQ ID NO: 7 - DNA, 17 bp, Artificial Sequence
  synthetically generated oligonucleotide probe
ccgggaaggtgtgcacg

SEQ ID NO: 8 - DNA, 24 bp, Artificial Sequence
  synthetically generated oligonucleotide probe
agagtcctgaggactgtaggacag

SEQ ID NO: 9 - DNA, 19 bp, Artificial Sequence
  synthetically generated oligonucleotide probe
accacgctgctgagggagt

SEQ ID NO: 10 - DNA, 17 bp, Artificial Sequence
  synthetically generated oligonucleotide probe
tgctggagggcacggtc

SEQ ID NO: 11 - DNA, 23 bp, Artificial Sequence
  synthetically generated oligonucleotide probe
gccatccacgtaccagttgaact

SEQ ID NO: 12 - DNA, 22 bp, Artificial Sequence
  synthetically generated oligonucleotide probe
tcttggcattatgcacctccac

SEQ ID NO: 13 - DNA, 23 bp, Artificial Sequence
  synthetically generated oligonucleotide probe
tttggagatggttttctcgatgg

SEQ ID NO: 14 - DNA, 17 bp, Artificial Sequence
  synthetically generated oligonucleotide probe
cggggctgccctttggc

SEQ ID NO: 15 - DNA, 22 bp, Artificial Sequence
  synthetically generated oligonucleotide probe
cagggtgtacacctgtggctct

SEQ ID NO: 16 - DNA, 20 bp, Artificial Sequence
  synthetically generated oligonucleotide probe
catctcctcctgggatgggg

SEQ ID NO: 17 - DNA, 22 bp, Artificial Sequence
  synthetically generated oligonucleotide probe
tcaggctgacctggttcttggt

SEQ ID NO: 18 - DNA, 20 bp, Artificial Sequence
  synthetically generated oligonucleotide probe
gaagcctttgaccaggcagg

SEQ ID NO: 19 - DNA, 18 bp, Artificial Sequence
  synthetically generated oligonucleotide probe
ggcgatgtcgctggggta

SEQ ID NO: 20 - DNA, 21 bp, Artificial Sequence
  synthetically generated oligonucleotide probe
cccattgctctcccactccac

SEQ ID NO: 21 - DNA, 22 bp, Artificial Sequence
  synthetically generated oligonucleotide probe
tcttgtagttgtcctccggctg

SEQ ID NO: 22 - DNA, 17 bp, Artificial Sequence
  synthetically generated oligonucleotide probe
cagcacgggaggcgtgg

SEQ ID NO: 23 - DNA, 21 bp, Artificial Sequence
  synthetically generated oligonucleotide probe
gaagaaggagccgtcggagtc

SEQ ID NO: 24 - DNA, 22 bp, Artificial Sequence
  synthetically generated oligonucleotide probe
ccacggttagcctgctgtagag

SEQ ID NO: 25 - DNA, 21 bp, Artificial Sequence
  synthetically generated oligonucleotide probe
cctcctgccacctgctcttgt

SEQ ID NO: 26 - DNA, 23 bp, Artificial Sequence
  synthetically generated oligonucleotide probe
cacggagcatgagaagacattcc

SEQ ID NO: 27 - DNA, 21 bp, Artificial Sequence
  synthetically generated oligonucleotide probe
gttgtgcagagcctcatgcat

SEQ ID NO: 28 - DNA, 25 bp, Artificial Sequence
  synthetically generated oligonucleotide probe
gggagaggctcttctgtgtgtagtg

SEQ ID NO: 29 - DNA, 24 bp, Artificial Sequence
  synthetically generated oligonucleotide probe
gaccatatttggactcaactctct

SEQ ID NO: 30 - DNA, 18 bp, Artificial Sequence
  synthetically generated oligonucleotide probe
ggcatgatgggcatgggg

SEQ ID NO: 31 - DNA, 18 bp, Artificial Sequence
  synthetically generated oligonucleotide probe
ccccaggaactcaggtgc

SEQ ID NO: 32 - DNA, 22 bp, Artificial Sequence
  synthetically generated oligonucleotide probe
ggaacaggaagactgatggtcc

(19) United States
(12) Patent Application Publication
(10) Pub. No.: US 2015/0232935 A1
(43) Pub. Date: Aug. 20, 2015

(54) METHODS FOR DIAGNOSING IGG4-RELATED DISEASE

(71) Applicants: The General Hospital Corporation, Boston, MA (US); Affymetrix, Inc., Santa Clara, CA (US)

(72) Inventors: Vikram Deshpande, Belmont, MA (US); Manoj Gandhi, Dublin, CA (US); Quan Nguyen, San Ramon, CA (US); Yunqing MA, San Jose, CA (US); David T. Ting, Dover, MA (US); Miguel Rivera, Cambridge, MA (US); Nicolo Riggi, Boston, MA (US)

(21) Appl. No.: 14/622,219

(22) Filed: Feb. 13, 2015

Related U.S. Application Data
(60) Provisional application No. 61/940,179, filed on Feb. 14, 2014.

(51) Int. Cl. C12Q 1/68; C07K 16/28

(52) U.S. Cl. CPC C12Q 1/6883; C07K 16/2887; C07K 2317/24; A61K 2039/505

(57) ABSTRACT

Methods for diagnosing and treating IgG4-related disease (IgG4-RD), e.g., based on detecting levels of IgG4 mRNA, preferably using a branched DNA assay.

### CLAIM OF PRIORITY

This application claims the benefit of U.S. Provisional Patent Application Ser. No. 61/940,179, filed on Feb. 14, 2014. The entire contents of the foregoing are hereby incorporated by reference.

### TECHNICAL FIELD

The present application relates to methods for diagnosing and treating IgG4-related disease (IgG4-RD), e.g., based on levels of IgG4 mRNA.

### BACKGROUND

Presence of a mass in any tissue can be broadly classified as being either of inflammatory or neoplastic origin, which are histologically distinct from each other. IgG4-related disease (IgG4-RD) is unique clinical condition where an inflammatory lesion closely resembles a tumor and hence is referred to as a pseudotumorous or a tumefactive lesion. IgG4-related disease is recognized now as a unique clinicopathologic entity characterized by tumefactive, fibroinflammatory lesions, the infiltration of IgG4-positive plasma cells into affected tissues, and often elevated concentrations of IgG4 in serum.¹ The most common gastrointestinal manifestations include autoimmune pancreatitis and IgG4-related sclerosing cholangitis.^{2 3} Although the diagnosis of IgG4-related disease is based on a constellation of clinical, radiological, and pathologic findings, histopathology is the gold standard for diagnosis.^{1, 4, 5} The histologic hallmarks include a dense lymphoplasmacytic infiltrate, storiform-type fibrosis, and obliterative phlebitis.⁵ However, a definitive diagnosis of IgG4-related disease also requires the presence of elevated numbers of IgG4-positive plasma cells. This can be problematic, because IgG4-positive plasma cells are also identified in a wide array of inflammatory and neoplastic diseases.⁶ In an attempt to improve the specificity of this test, a recent consensus document also requires the presence of a ratio of IgG4- to IgG-bearing plasma cells greater than 40%.⁵

Although the diagnosis of IgG4-related disease should not be based solely on the presence of elevated numbers of IgG4-bearing plasma cells, no firm diagnosis can be established without the accurate quantification of the numbers of IgG4- and IgG-bearing plasma cells in tissue. Unfortunately, immunohistochemical tests for immunoglobulins are associated with high background signal, which often makes quantitative analysis difficult. This difficulty is compounded further by the fact that the calculation of a ratio requires the enumeration of both IgG4- and IgG-bearing plasma cells, and a strong background signal on either preparation precludes this analysis. Needle biopsies from the liver and pancreas are particularly prone to this artifact.

### SUMMARY

The present invention is based, at least in part, on the development of methods for accurately diagnosing and optionally treating IgG4-related disease (IgG4-RD), e.g., based on detecting levels of IgG4 mRNA. The RNA-ISH platform presented here provides an alternative to immunohistochemistry for the diagnosis of IgG4-related disease. In situ hybridization is particularly valuable in situations where the background signal makes counting positive cells arduous or impossible. The in situ hybridization platform also offers additional value since there is a more robust separation between IgG4-RD cases and its mimics on the basis of the IgG4:total IgG ratio. Finally, the detection of IgG4 signals in lymphocytes may in part explain the dramatic response to anti-CD20 therapy in IgG4 related disease, thus the quantitation of similar signals in this and other diseases may be of diagnostic value.

Thus, there are provided herein methods for diagnosing a tumefactive lesion associated with an IgG4-related disease (IgG4-RD) in a subject who has a mass. The methods include contacting a sample comprising plasma cells from the mass with one or more polynucleotide probes that bind specifically to IgG4 mRNA in situ, and one or more polynucleotide probes that bind specifically to IgG mRNA in situ; detecting binding of the probes to IgG4 mRNA and IgG mRNA in plasma cells in the sample, to determine numbers of IgG4-plasma cells and IgG-plasma cells; calculating a ratio of IgG4-plasma cells to IgG-plasma cells; and identifying a sample in which the ratio of IgG4-plasma cells to IgG-plasma cells is above a threshold as a tumefactive lesion associated with an IgG4-RD, or identifying a sample in which the IgG4-plasma cells to IgG-plasma cells ratio is below a threshold as not being a tumefactive lesion associated with an IgG4-RD.

There are also provided herein methods for selecting a treatment for a subject who has a mass. The methods include contacting a sample comprising plasma cells from the mass with one or more polynucleotide probes that bind specifically to IgG4 mRNA in situ, and one or more polynucleotide probes that bind specifically to IgG mRNA in situ; detecting binding of the probes to IgG4 mRNA and IgG mRNA in plasma cells in the sample, to determine numbers of IgG4-plasma cells and IgG-plasma cells; calculating a ratio of IgG4-plasma cells to IgG-plasma cells; and identifying a sample in which the ratio of IgG4-plasma cells to IgG-plasma cells is above a threshold as a tumefactive lesion associated with an IgG4-RD, and selecting for the subject a treatment for an IgG4-RD; or identifying a sample in which the IgG4-plasma cells to IgG-plasma cells ratio is below a threshold as not being a tumefactive lesion associated with an IgG4-RD.

There are also provided herein methods for treating a subject who has a mass. The methods include contacting a sample comprising plasma cells from the mass with one or more polynucleotide probes that bind specifically to IgG4 mRNA in situ, and one or more polynucleotide probes that bind specifically to IgG mRNA in situ; detecting binding of the probes to IgG4 mRNA and IgG mRNA in plasma cells in the sample, to determine numbers of IgG4-plasma cells and IgG-plasma cells; calculating a ratio of IgG4-plasma cells to IgG-plasma cells; and identifying a sample in which the ratio of IgG4-plasma cells to IgG-plasma cells is above a threshold as a tumefactive lesion associated with an IgG4-RD, and administering to the subject a treatment for an IgG4-RD; or identifying a sample in which the IgG4-plasma cells to IgG-plasma cells ratio is below a threshold as not being a tumefactive lesion associated with an IgG4-RD.

There are also provided herein methods for making a differential diagnosis between a mass that is a tumefactive lesion associated with an IgG4-RD or a mass that is not a tumefactive lesion associated with an IgG4-RD in a subject who has a mass. The methods include contacting a sample comprising plasma cells from the mass with one or more polynucleotide probes that bind specifically to IgG4 mRNA in situ, and one or more polynucleotide probes that bind specifically to IgG mRNA in situ; detecting binding of the probes to IgG4 mRNA and IgG mRNA in plasma cells in the sample, to determine numbers of IgG4-plasma cells and IgG-plasma cells; calculating a ratio of IgG4-plasma cells to IgG-plasma cells; and diagnosing a subject who has a mass in which the ratio of IgG4-plasma cells to IgG-plasma cells is above a threshold as having a tumefactive lesion associated with an IgG4-RD, or diagnosing a subject with a mass in which the ratio of IgG4-plasma cells to IgG-plasma cells is below a threshold as having a tumefactive lesion not associated with an IgG4-RD.

In some embodiments, two or more (e.g., a plurality of) polynucleotide probes that bind specifically to IgG4 mRNA and/or two or more (e.g., a plurality of) polynucleotide probes that bind specifically to IgG mRNA are used. In some embodiments wherein only a single polynucleotide probe is used, e.g., a single probe that binds specifically to IgG4 mRNA or to IgG mRNA, more signal might need to be generated, so an appropriate label and/or greater amplification of that label can be used. For example, in embodiments using bDNA with a single label extender, a larger “tree” can be used than in methods using multiple label extenders and label probe systems.

In some embodiments, the methods include identifying a mass that is not a tumefactive lesion associated with an IgG4-RD as being a neoplastic tumor; optionally determining the tissue of origin of the tumor; and optionally selecting and/or administering to the subject a treatment for cancer, e.g., a treatment for a cancer of the tissue of origin.

In some embodiments, the methods include determining whether the IgG4-RD is Autoimmune pancreatitis; Eosinophilic angiocentric fibrosis; Fibrosing mediastinitis; Hypertrophic pachymeningitis; Idiopathic hypocomplementemic tubulointerstitialnephritis with extensive tubulointerstitial deposits; Inflammatory aortic aneurysm; Inflammatory pseudotumor; Küttner's tumor (chronic sclerosing sialadenitis); Mediastinal fibrosis; Mikulicz's syndrome; Multifocal fibrosclerosis; Periaortitis and periarteritis; Retroperitoneal fibrosis (Ormond's disease); Riedel's thyroiditis; Sclerosing mesenteritis; Sclerosing pancreatitis; or Sclerosing cholangitis, e.g., based on the location of the mass in the subject's body.

In some embodiments, the sample is a biopsy sample obtained from the subject, and preferably wherein the sample comprises a plurality of individually identifiable cells. In some embodiments, the sample has been fixed, preferably with formalin, optionally embedded in a matrix, e.g., paraffin, e.g., a formaldehyde-fixed, paraffin-embedded (FFPE) clinical sample, and wherein the sample has been sliced into sections.

In some embodiments, the one or more polynucleotide probes that bind specifically to IgG4 mRNA in situ, and the one or more polynucleotide probes that bind specifically to IgG mRNA in situ, are both applied to a single section from the sample. In some embodiments, the one or more polynucleotide probes that bind specifically to IgG4 mRNA in situ, and the one or more polynucleotide probes that bind specifically to IgG mRNA in situ, are applied to consecutive sections from the sample.

In some embodiments, binding of the probes to IgG4 mRNA and IgG mRNA is detected using imaging, e.g., microscopy, e.g., bright-field or fluorescence microscopy, and preferably wherein at least three high power fields (HPF) (e.g., viewed using a 40× objective) in the mass are analyzed to determine the number of IgG4-positive and IgG-positive cells.

In some embodiments, the methods include detecting binding of the probes to IgG4 mRNA and IgG mRNA in the cytoplasm of the plasma cells in the sample, to determine numbers of IgG4-plasma cells and IgG-plasma cells.

In some embodiments, the methods include detecting levels of IgG4 in serum, wherein the presence of elevated IgG4 in serum, plus the presence of the ratio of IgG4-plasma cells to IgG-plasma cells that is above a threshold, indicates that the subject has a tumefactive lesion associated with an IgG4-RD.

In some embodiments, the methods include evaluating the morphology of the cells in the sample, and (i) identifying a sample having abundant inflammatory cells, mainly plasma cells, fibrosis and obliterative phlebitis, and a ratio of IgG4-plasma cells to IgG-plasma cells is above a threshold as being from a early- or mid-stage tumefactive lesion associated with an IgG4-RD; (ii) identifying a sample having extensive fibrosis with few plasma cell inflammatory infiltrates and ratio of IgG4-plasma cells to IgG-plasma cells is above a threshold as being from an advanced tumefactive lesion associated with an IgG4-RD; or (iii) identifying a sample having abundant inflammatory cells, mainly plasma cells, and fibrosis, and ratio of IgG4-plasma cells to IgG-plasma cells below a threshold, as being from a neoplastic tumor.

In some embodiments, the methods include identifying a sample in which the ratio of IgG4-plasma cells to IgG-plasma cells is above a threshold; detecting IgKC and IgLC mRNA in the cells in the sample; and identifying a sample that has IgKC/IgLC clonality as being a IgG4 related lymphoma, or identifying a sample that does not have IgK/IgL clonality as being a tumefactive lesion associated with an IgG4-RD.

In some embodiments, the one or more probes comprise probes that bind to a plurality of target regions in the IgG4 or IgG mRNA.

In some embodiments, the one or more probes that bind to IgG4 mRNA bind to a non-homologous constant region of Homo sapiens Ig heavy chain gamma4, e.g., within the sequence CAGCTTGGGCACGAAGACCTACACCTGCAACGTAGATCACAAGCCCAGCAACAC CAAGGTGGACAAGAGAGTTGAGTCCAAATATGGTCCCCCATGCCCATCATGCCCA GCACCTGAGTTCCTGGGGGACCATCAGTCTTCCTGTTCCCCCCAAAACC (SEQ ID NO:1); and/or

the one or more probes that bind to IgG mRNA bind to a conserved constant region of the four Ig heavy gamma sequences, e.g., within the double-underlined portions of the following sequence:

(SEQ ID NO: 2)

GCAAGCTTCAAGGGCCCATCGGTCTTCCCCCTGGTGCCCTGCTCCAGGAG
CACCTCCGAGAGCACAGCCGCCCTGGGCTGCCTGGTCAAGGACTACTTCC
CCGAACCGGTGACGGTGTCGTGGAACTCATGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACGAAGACCTACACCTGCA
ACGTAGATCACAAGCCCAGCAACACCAAGGTGGACAAGAGAGTTGAGTCC
AAATATGGTCCCCCATGCCCATCATGCCCAGCACCTGAGTTCCTGGGGGG
ACCATCAGTCTTCCTGTTCCCCCCAAAACCCAAGGACACTCTCATGATCT
CCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCA
GGGTCCTCACCGTCCTGCACCAGGACTGGCTGAACGGTAAGGAGTACAAG
TGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCAT
CCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCC
GGAGGACAACTACAAGACCACGCCTCCCGTGCTGGACTCCGACGGCTCCT
TCTTCCTCTACAGCAGGCTAACCGTGGACAAGAGCAGGTGGCAGGAGGGG
AATGTCTTCTCATGCTCCGTGATGCATGAGGCTCTGCACAACCACTACAC
ACAGAAGAGCCTCTCCCTGTCTCCGGGTAAA.

In some embodiments, the one or more probes that bind to IgG4 mRNA comprises probes that hybridize to at least 2, 3, 4, 5, 6, 7, or 8 different target sequences within the non-homologous constant region of Homo sapiens Ig heavy chain gamma4, e.g., within the sequence CAGCTTGGGCACGAAGACCTACACCTGCAACGTAGATCACAAGCCCAGCAACAC CAAGGTGGACAAGAGAGTTGAGTCCAAATATGGTCCCCCATGCCCATCATGCCCA GCACCTGAGTTCCTGGGGGACCATCAGTCTTCCTGTTCCCCCCAAAACC (SEQ ID NO:XX); and/or the one or more probes that bind to IgG mRNA comprises probes that hybridize to at least 2, 3, 4, 5, 6, 7, 8, 9, 10, 11, 12, 13, 14, 15, 16, 17, 18, 19, 20, 21, 22, 23, 24, 25, or 26 different target sequences within the bind to a conserved constant region of the four Ig heavy gamma sequences, e.g., within the double-underlined portions of the following sequence:

(SEQ ID NO: 2)

GCAAGCTTCAAGGGCCCATCGGTCTTCCCCCTGGTGCCCTGCTCCAGGAG
CACCTCCGAGAGCACAGCCGCCCTGGGCTGCCTGGTCAAGGACTACTTCC
CCGAACCGGTGACGGTGTCGTGGAACTCATGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACGAAGACCTACACCTGCA
ACGTAGATCACAAGCCCAGCAACACCAAGGTGGACAAGAGAGTTGAGTCC
AAATATGGTCCCCCATGCCCATCATGCCCAGCACCTGAGTTCCTGGGGGG
ACCATCAGTCTTCCTGTTCCCCCCAAAACCCAAGGACACTCTCATGATCT
CCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCA
GGGTCCTCACCGTCCTGCACCAGGACTGGCTGAACGGTAAGGAGTACAAG
TGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCAT
CCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCC
GGAGGACAACTACAAGACCACGCCTCCCGTGCTGGACTCCGACGGCTCCT
TCTTCCTCTACAGCAGGCTAACCGTGGACAAGAGCAGGTGGCAGGAGGGG
AATGTCTTCTCATGCTCCGTGATGCATGAGGCTCTGCACAACCACTACAC
ACAGAAGAGCCTCTCCCTGTCTCCGGGTAAA

In some embodiments, the binding of the probes to IgG4 mRNA and IgG mRNA is detected using one or more labels that are directly or indirectly bound to the polynucleotide probes.

In some embodiments, the binding of the probes to IgG4 mRNA is detected using branched nucleic acid signal amplification.

In some embodiments, the probes are branched DNA probes.

In some embodiments, the methods include contacting the sample with a plurality of probes that comprises one or more label extender probes that bind to one or more target regions in the IgG4 mRNA; hybridizing one or more pre-amplifier probes to the one or more label extender probes; hybridizing one or more amplifier probes to the pre-amplifier probes; and hybridizing one or more label probes to the one or more amplifier probes.

In some embodiments, the methods include contacting the sample with a plurality of probes that comprises one or more label extender probes that bind to one or more target regions in the IgG mRNA; hybridizing one or more pre-amplifier probes to the one or more label extender probes; hybridizing one or more amplifier probes to the pre-amplifier probes; and hybridizing one or more label probes to the one or more amplifier probes.

In some embodiments, the label probes are conjugated to an enzyme, and binding of the probe is detected using a chromogen substrate with the enzyme.

In some embodiments, the label probes are conjugated to a fluorophore, and binding of the probe is detected by observation of emissions from the fluorophore after illumination suitable to excite the fluorophore.

In some embodiments, the one or more polynucleotide probes that bind specifically to IgG4 mRNA in situ, and the one or more polynucleotide probes that bind specifically to IgG mRNA in situ, are both applied to a single section from the sample, and binding of the one or more polynucleotide probes to IgG4 is detected using a first detectable signal, and binding of the one or more polynucleotide probes to IgG is detected using a second detectable signal.

In some embodiments, the methods include contacting a sample comprising tissue from the tumor with one or more polynucleotide probes that bind specifically to mRNA encoding a housekeeping gene (HKG) in situ;

detecting binding of the one or more probes to HKG mRNA, and
selecting for further analysis a sample in which binding of the one or more probes to the HKG mRNA is detected, or rejecting a sample in which binding of the one or more probes to the HKG mRNA is not detected. In some embodiments, the binding of the probes to IgG4 mRNA, IgG mRNA, or HKG mRNA is detected using branched nucleic acid signal amplification. In some embodiments, the probes are branched DNA probes.

In some embodiments, the methods include contacting the sample with a plurality of probes that comprises one or more label extender probes that bind to a plurality of target regions in the IgG4, IgG, or HKG mRNA; hybridizing one or more pre-amplifier probes to the one or more label extender probes; hybridizing one or more amplifier probes to the pre-amplifier; and hybridizing one or more label probes to the one or more amplifier probes.

In some embodiments, the one or more polynucleotide probes that bind specifically to IgG4 mRNA in situ and the one or more polynucleotide probes that bind specifically to IgG mRNA in situ are applied to consecutive sections from the sample, the label probes are conjugated to an enzyme, binding of the IgG4 probes to IgG4 mRNA and IgG probes to IgG mRNA is detected using a first chromogen substrate for the enzyme, and binding of the HKG probes to HKG mRNA is detected using a second chromogen substrate for the enzyme.

In some embodiments, the one or more polynucleotide probes that bind specifically to IgG4 mRNA in situ and the one or more polynucleotide probes that bind specifically to IgG mRNA in situ are applied to consecutive sections from the sample, the label probes are conjugated to a fluorophore, binding of the IgG4 probes to IgG4 mRNA and IgG probes to IgG mRNA is detected using a first fluorophore, and binding of the HKG probes to HKG mRNA is detected using a second fluorophore.

In some embodiments, the one or more polynucleotide probes that bind specifically to IgG4 mRNA in situ and the one or more polynucleotide probes that bind specifically to IgG mRNA in situ are both applied to a single section from the sample, the label probes are conjugated to an enzyme, binding of the IgG4 probes to IgG4 mRNA is detected using a first chromogen substrate for the enzyme, IgG probes to IgG mRNA is detected using a second chromogen substrate for the enzyme, and binding of the HKG probes to HKG mRNA is detected using a third chromogen substrate for the enzyme.

In some embodiments, the one or more polynucleotide probes that bind specifically to IgG4 mRNA in situ and the one or more polynucleotide probes that bind specifically to IgG mRNA in situ are both applied to a single section from the sample, the label probes are conjugated to a fluorophore, binding of the IgG4 probes to IgG4 mRNA is detected using a first fluorophore, binding of the IgG probes to IgG mRNA is detected using a second fluorophore, and binding of the HKG probes to HKG mRNA is detected using a third fluorophore.

The following definitions can be understood with reference to FIG. 1D. A “label extender” is a polynucleotide that is capable of hybridizing to both a nucleic acid analyte and also to at least a portion of a label probe system. A label extender typically has a first polynucleotide sequence L-1, which is complementary to a polynucleotide sequence of the nucleic acid analyte, and a second polynucleotide sequence L-2, which is complementary to a polynucleotide sequence of the label probe system (e.g., L-2 can be complementary to a polynucleotide sequence of a preamplifier, amplifier, a label probe, or the like). The label extender is preferably a single-stranded polynucleotide. Non-limiting examples of label extenders in various configurations and orientations are disclosed within, e.g., U.S. Published Patent Application No. 2012/0052498 (including but not limited to those depicted within FIGS. 10A and 10B).

A “label probe system” comprises one or more polynucleotides that collectively comprise one or more label probes which are capable of hybridizing, directly or indirectly, to one or more label extenders in order to provide a detectable signal from the labels that are associated or become associated with the label probes. Indirect hybridization of the one or more label probes to the one or more label extenders can include the use of amplifiers, or the use of both amplifiers and preamplifiers, within a particular label probe system. Label probe systems can also include two or more layers of amplifiers and/or preamplifiers to increase the size of the overall label probe system and the total number of label probes (and therefore the total number of labels that will be used) within the label probe system. The configuration of the label probe system within a particular embodiment is typically designed in the context of the overall assay, including factors such as the amount of signal required for reliable detection of the target analyte in the assay, the particular label being used and its characteristics, the number of label probes needed to provide the desired level of sensitivity, maintaining the desired balance of specificity and sensitivity of the assay, and other factors known in the art.

An “amplifier” is a polynucleotide comprising one or more polynucleotide sequences A-1 and one more polynucleotide sequences A-2. The one or more polynucleotide sequences A-1 may or may not be identical to each other, and the one or more polynucleotide sequences A-2 may or may not be identical to each other. Within label probe systems utilizing amplifiers and label probes, polynucleotide sequence A-1 is typically complementary to polynucleotide sequence L-2 of the one or more label extenders, and polynucleotide sequence A-2 is typically complementary to polynucleotide sequence LP-1 of the label probes. Within label probe systems utilizing amplifiers, preamplifiers and label probes, polynucleotide sequence A-1 is typically complementary to polynucleotide sequence P-2 of the one or more preamplifiers, and polynucleotide sequence A-2 is typically complementary to polynucleotide sequence LP-1 of the label probes. Amplifiers can be, e.g., linear or branched polynucleotides.

A “preamplifier” is a polynucleotide comprising one or more polynucleotide sequences P-1 and one or more polynucleotide sequences P-2. The one or more polynucleotide sequences P-1 may or may not be identical to each other, and the one or more polynucleotide sequences P-2 may or may not be identical to each other. When one or more preamplifiers are utilized within a label probe system, polynucleotide sequence P-1 is typically complementary to polynucleotide sequence L-2 of the label extenders, and polynucleotide sequence P-2 is typically complementary to polynucleotide sequence A-1 of the one or more amplifiers. Preamplifiers can be, e.g., linear or branched polynucleotides.

A “label probe” is a single-stranded polynucleotide that comprises a label (or optionally that is configured to bind, directly or indirectly, to a label) to directly or indirectly provide a detectable signal. The label probe typically comprises a polynucleotide sequence LP-1 that is complementary to a polynucleotide sequence within the label probe system, or alternatively to the one or more label extenders. For example, in different embodiments, label probes may hybridize to either an amplifier and/or preamplifier of the label probe system, while in other embodiments where neither an amplifier nor preamplifier is utilized, a label probe may hybridize directly to a label extender.

A “label” is a moiety that facilitates detection of a molecule. Common labels in the context of the present invention include fluorescent, luminescent, light-scattering, and/or colorimetric labels. Suitable labels include enzymes and fluorescent moieties, as well as radionuclides, substrates, cofactors, inhibitors, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Labels include the use of enzymes such as alkaline phosphatase that are conjugated to an polynucleotide probe for use with an appropriate enzymatic substrate, such as fast red or fast blue, which is described within U.S. Pat. Nos. 5,780,227 and 7,033,758. Alternative enzymatic labels are also possible, such as conjugation of horseradish peroxidase to polynucleotide probes for use with 3,3′-Diaminobenzidine (DAB). Many labels are commercially available and can be used in the context of the invention.

The term “polynucleotide” encompasses any physical string of monomer units that correspond to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the polynucleotide can be deoxyribonucleotides, ribonucleotides or nucleotide analogs, can be natural or non-natural (e.g., locked nucleic acids, isoG or isoC nucleotides), and can be unsubstituted, unmodified, substituted or modified. The nucleotides can be linked by phosphodiester bonds, or by phosphorothioate linkages, methylphosphonate linkages, boranophosphate linkages, or the like. Polynucleotides can additionally comprise non-nucleotide elements such as labels, quenchers, blocking groups, or the like. Polynucleotides can be, e.g., single-stranded, partially double-stranded or completely double-stranded.

The term “probe” refers to a non-analyte polynucleotide.

Two polynucleotides “hybridize” when they associate to form a stable duplex, e.g., under relevant assay conditions. Polynucleotides hybridize due to a variety of well characterized physicochemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, New York).

The term “complementary” refers to a polynucleotide that forms a stable duplex with its complement sequence under relevant assay conditions. Typically, two polynucleotide sequences that are complementary to each other have mismatches at less than about 20% of the bases, at less than about 10% of the bases, preferably at less than about 5% of the bases, and more preferably have no mismatches.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-B: Schematic representations of exemplary 1-plex tissue assay using a bDNA platform.

FIG. 1C: Schematic representation of an exemplary 2-plex tissue assay using a bDNA platform.

FIG. 1D: Schematic illustration of an exemplary bDNA amplification scheme.

FIG. 1E. In situ hybridization for IgG4 performed on an ampullary biopsy reveals bright reactivity within plasma cells with virtually no staining of the background tissue.

FIGS. 2A-D. IgG4 related pulmonary disease. Immunohistochemical stains for IgG4 (A) and IgG (B) showed strong background signal precluding a quantitative analysis. The control samples (tonsil) placed on the same slide did not suffer from this artifact. In situ hybridization stain for IgG4 (C) and IgG (D).

FIGS. 3A-B. Error bars comparing the IgG4 counts and the IgG4 to IgG ratio on the immunohistochemical and in situ hybridization platforms.

FIGS. 4A-D. IgG4 related disease of the pleural cavity. Both the immunohistochemical stains (A and B) as well as the in situ hybridization stains (C and D) performed well.

FIGS. 5A-B. In situ hybridization stain for IgG4 (A). The plasma cells show a strong signal. However signal was also identified in the majority of lymphocytes. The signal within the lymphocytes is however significantly less than that seen in the plasma cells. In situ hybridization for IgG (B). The intensely positive cells represent plasma cells. The lymphocytes also show intracytoplasmic stain. However the intensity of reactivity within lymphocytes is significantly more than the IgG4 stain, a finding related to the probe design.

FIG. 6. In situ hybridization for IgG4. The intensely positive cells represent plasma cells. A weaker signal is seen in the mature lymphocytes.

FIGS. 7A-B are each images showing a set of three fields stained for IgG (top row) or IgG4 (bottom row). 7A, sample from a subject with non-IgG4 disease; 7B, sample from a subject with IgG4-related disease.

FIGS. 8A-B. Schematic illustrations of exemplary algorithms for differential diagnosis of IgG4-RD from non-IgG4-RD.

### DETAILED DESCRIPTION

IgG4-RD is a tumefactive fibroinflammatory lesion that is histologically characterized by dense inflammation, including blood vessels, accompanied by fibrosis. Patients with IgG4-RD have elevated levels of IgG4-positive plasma cells in the tissues. This may or may not be associated with an increase in serum IgG4 levels.

Recent medical literature suggests that IgG4-RD can involve almost any organ (Mahajan et al., Annu Rev. Pathol. Mech. Dis. 2014. 9:315-47 (2014; Epub ahead of print Oct. 2, 2013); Stone et al., N Engl J Med. 366(6):539-51 (2012)). Diseases including autoimmune pancreatitis, Mikulicz's syndrome (lacrimal and salivary gland), Kuttner's tumor (submandibular salivary gland), Riedel's thyroiditis, and retroperitoneal fibrosis (Ormond's disease), which have been identified as unique medical conditions in the past, are now considered part of the spectrum of IgG4-RD (see Table 1). Consequently, better understanding of this disease has led to other conditions being reclassified as IgG4-RD.

The diagnosis of IgG4 related disease relies on a constellation of findings: history and physical examination, imaging, elevated serum IgG4 concentrations, the presence of multi-organ involvement, and the histopathological evaluation of affected tissue. Histopathology has emerged as the gold standard for diagnosis in this disease, and the demonstration of elevated numbers of IgG4-positive plasma cells as well as an elevated IgG4 to IgG ratio constitutes a critical element of this analysis. However, standard immunohistochemical preparations for immunoglobulins are often associated with marked nonspecific staining—“background signal”—that precludes quantitative evaluation. Needle biopsies from pancreatic and hepatic lesions are particularly prone to these staining artifacts.

Distinguishing IgG4-RD from disorders that mimic it frequently, e.g., malignancy, granulomatosis with polyangiitis, sarcoidosis, and a host of other conditions, relies heavily on the demonstration of elevated numbers of IgG4-positive cells and elevated IgG4 to IgG ratios in tissue. Misdiagnoses may lead to inappropriate treatments or procedures (e.g., Whipple procedures), and diagnostic delays may close the already narrow window for surgical resection, particularly for malignancies of the pancreatobiliary system.

IgG4-RD

As noted above, IgG4-RD can involve almost any organ. Common sites of involvement are the pancreas, hepatobiliary tract, salivary gland, orbit, and lymph node; less common are lesions of the aerodigestive tract, lung, aorta, mediastinum, retroperitoneum, soft tissue, skin, central nervous system, breast, kidney, and prostate.

Before the present invention, a diagnosis of IgG4-RD was typically made based on the presence of two factors: (1) elevations in serum IgG4 concentrations, and (2) a set of unique histopathological characteristics including lymphoplasmacytic infiltrate, storiform fibrosis, obliterative phlebitis, and mild to moderate tissue eosinophilia (5, 6). Storiform fibrosis is associated with a pattern seen on histological examination under low-power light microscopy that includes irregular, loosely arranged whorls, similar to a straw blanket. Obliterative phlebitis is severe inflammation of a vein that results in fibrosis and permanent closure of the vessel.

IgG4-RD is most common in males of middle age or older. Table 1 lists a number of the IgG4-RD spectrum conditions.

TABLE 1
IgG4-RD Spectrum Conditions

| Condition | Affects: |
|---|---|
| Autoimmune pancreatitis | Pancreas |
| Eosinophilic angiocentric | Orbits, upper respiratory tract |
| fibrosis | |
| Fibrosing mediastinitis | Mediastinum |
| Hypertrophic pachymeningitis | Dura mater |
| Idiopathic hypocomplementemic | Kidney |
| tubulointerstitialnephritis | |
| with extensive tubulointerstitial | |
| deposits | |
| Inflammatory aortic aneurysm | Aorta |
| Inflammatory pseudotumor | Orbits, lungs, kidneys, and other organs |
| Küttner's tumor | Submandibular glands |
| (chronic sclerosing sialadenitis) | |
| Mediastinal fibrosis | Mediastinum |
| Mikulicz's syndrome | Salivary and lacrimal glands |
| Multifocal fibrosclerosis | Orbits, thyroid gland, retroperitoneum, |
| | mediastinum, and other tissues and |
| | organs |
| Periaortitis and periarteritis | Aorta and large blood vessels |
| Retroperitoneal fibrosis | Retroperitoneum |
| (Ormond's disease) | |
| Riedel's thyroiditis | Thyroid |
| Sclerosing mesenteritis | Mesentery |
| Sclerosing pancreatitis | Pancreas |
| Sclerosing cholangitis | Bile duct, gallbladder, and salivary |
| | gland |

Methods of Detection and Diagnosis

Because IgG4-RD tends to form tumefactive lesions, patients are often suspected of having a malignancy. In light of the different treatments, an accurate diagnosis is crucial. However, the disease has been difficult to diagnose using standard methodology. For example, approximately 30% of IgG4-RD patients have normal serum IgG4 concentrations, despite the presence of classic histopathological and immunohistochemical findings indicative of IgG4-RD (Sah et al., Curr Opin Rheumatol 23:108-13 (2011)). Features detected using standard imaging technologies are generally nonspecific and do not permit reliable distinctions between IgG4-related disease and cancer (Stone et al., N Engl J Med. 366(6):539-51 (2012)).

Preferred embodiments include performing a semiquantitative ratiometric analysis of the proportion of IgG4-expressing plasma cells in comparison to IgG-expressing plasma cells. An IgG4/IgG ratio over a set threshold, e.g., over 20%, preferably over 30%, more preferably over 40%, or even more preferably over 50%, confirms a diagnosis of IgG4-RD (see Stone et al. (2012), for the use of a ratio of IgG4 to IgG of higher than 50% as evidence of IgG4-related disease). The caveat is that in the late phase of disease where there is severe fibrosis with few plasma cells, the test may not yield accurate information. The pattern of fibrosis and IgG4/IgG ratio are critical components in the diagnosis of IgG4-RD.

To overcome the known deficiencies of immunohistochemical approaches to providing a quantitative IgG4/IgG ratio, an in situ hybridization platform was used to estimate IgG4 counts and an IgG4:IgG ratio in 7 of the 22 IgG4-RD patients studied. A remarkable aspect of the RNA in situ hybridization platform is that the 19 cases in which the enumeration of IgG4-bearing plasma cells or IgG plasma cells or both proved unworkable because of strong background signal on immunohistochemistry were easily quantified on the in situ hybridization platform. On the immunohistochemical platform, lymph node tissue placed on the same slide did not show this staining artifact. Thus, it appears that certain tissue types, such as ampullary and pancreatic needle biopsies, are prone to a non-specific signal on immunohistochemistry. Thus IHC for IgG4 and IgG is often associated with high background signal, which makes definitive diagnosis challenging.

An in situ hybridization approach is able to overcome the problems associated with the current immunohistochemical platform since an immunohistochemical method for secreted proteins is invariably associated with intense nonspecific signal in adjacent tissue. However, there is significant homology between the 4 isoforms of IgG heavy chain gene, and only a sequence of 80 nucleotides in the hinge region is unique to the IgG4 isoform. Thus, the signal with conventional in situ hybridization assays would be relatively weak, and lack the bright reactivity necessary for quantitative analysis. The branched-chain amplification RNA-ISH platform presented herein allows for increased amplification and results in bright signals within plasma cells, as well as a slightly diminished but still easily visualized reactivity within mature-appearing lymphocytes.

In the performed studies, RNA-ISH stains for IgG4 and IgG were validated in a cohort of clinically and pathologically confirmed patients with IgG4-related disease. The control cohort was carefully chosen to include cases that often mimic IgG4-related disease in its clinical, serological, or histopathological features. This group included cases that showed elevated numbers of IgG4-bearing plasma cells as well as elevated IgG4: total IgG. The highly relevant control group broadens the clinical situations to which the present findings can be extrapolated.

In some embodiments, a differential diagnosis of IgG4-RD versus non-IgG4-RD can be made using the following criterion

| IgG4-RD | Non-IgG4-RD |
|---|---|
| 1. Lesions with abundant | 1. Lesions with abundant |
| inflammatory cells, | inflammatory cells, |
| mainly plasma cells, | mainly plasma cells, |
| fibrosis and obliterative | fibrosis. Plasma cells |
| phlebitis. | show intense cytoplasmic staining |
| 2. Advanced disease shows | 2. IgG4/IgG below a |
| extensive fibrosis | threshold, e.g., below |
| with few plasma cell | 20%, 30%, 40%, or 50%, |
| inflammatory infiltrate. | |
| 3. Serum IgG4 levels | |
| may be elevated | |
| 4. IgG4/IgG over a threshold, | |
| e.g., over 20%, 30%, 40%, | |
| or 50% | |

Detecting IgG and IgG4

The methods described herein that detect RNA in situ, e.g., in formalin fixed paraffin embedded material, fresh frozen tissue sections, fine needle aspirate biopsies, tissue microarrays, cells isolated from blood (including whole blood), bone marrow or sputum (such as samples prepared using centrifugation (such as with the CytoSpin Cytocentrifuge instrument (ThermoFisher Scientific, Waltham, Mass.) or smeared on a slide), blood smears on slides (including whole blood smears), and other sample types where the cellular morphology is sufficiently intact to allow the identification of samples with an IgG4/IgG ratio above a threshold, enable physicians to refine their diagnostic precision as well as provide novel prognostic and predictive biomarkers. In preferred embodiments, the sample will be taken from the mass, i.e., the fibroinflammatory tissue mass (which as described above can be present in various organs).

In some embodiments of the present methods, plasma cells, which can be identified by their intense cytoplasmic staining (e.g., numerous dots such that individual dots are not discernible at 4-40×) with IgG and/or IgG4 probes, are analyzed for the number of IgG4- and IgG-positive plasma cells using RNA ISH. For all cases the following are excluded from the analysis:

1. Staining outside of the cytoplasm of cells

2. Lymphocytes showing presence of nuclear staining on ISH

3. Lymphocytes showing less than 5 dots/cell in cytoplasm

4. Plasma Cells showing presence of nuclear staining on ISH

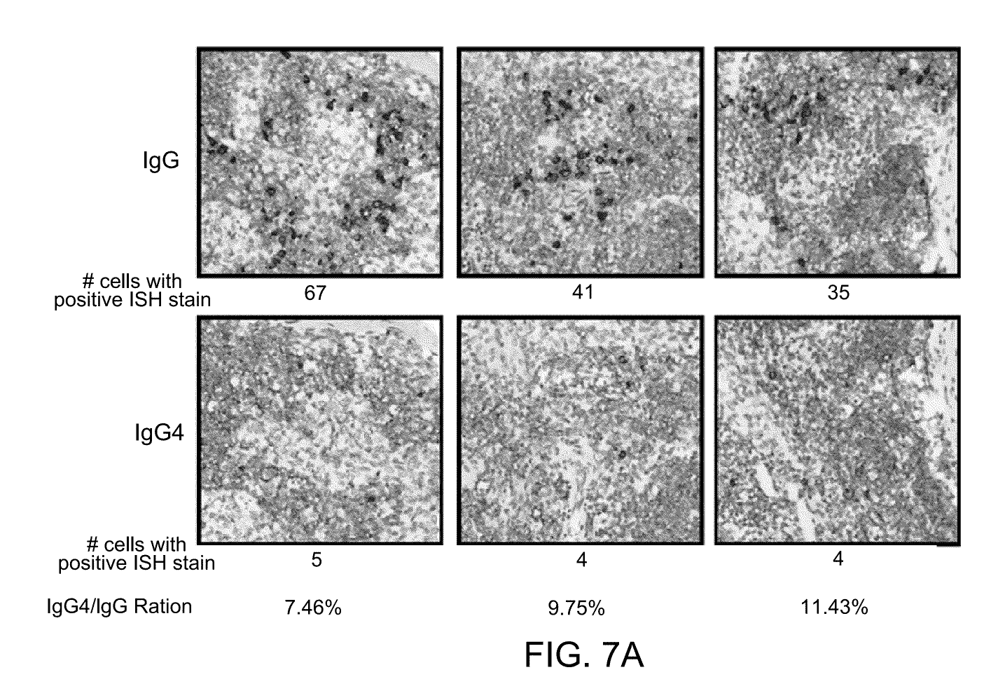

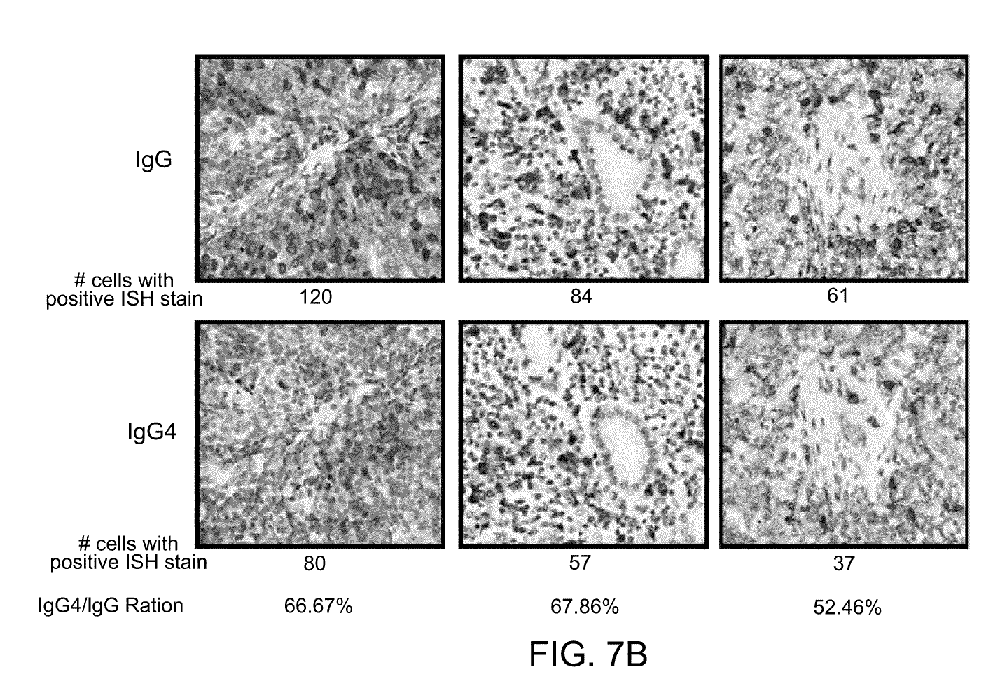

In preferred embodiments, at least three high power fields (HPF) (e.g., 40×) in the lesion are analyzed for the number of IgG4-positive (IgG4+) and IgG-positive (IgG+) plasma cells on the ISH. As shown in FIGS. 7A-B, the IgG+ and IgG4+ cells per field are counted, and the mean determined.

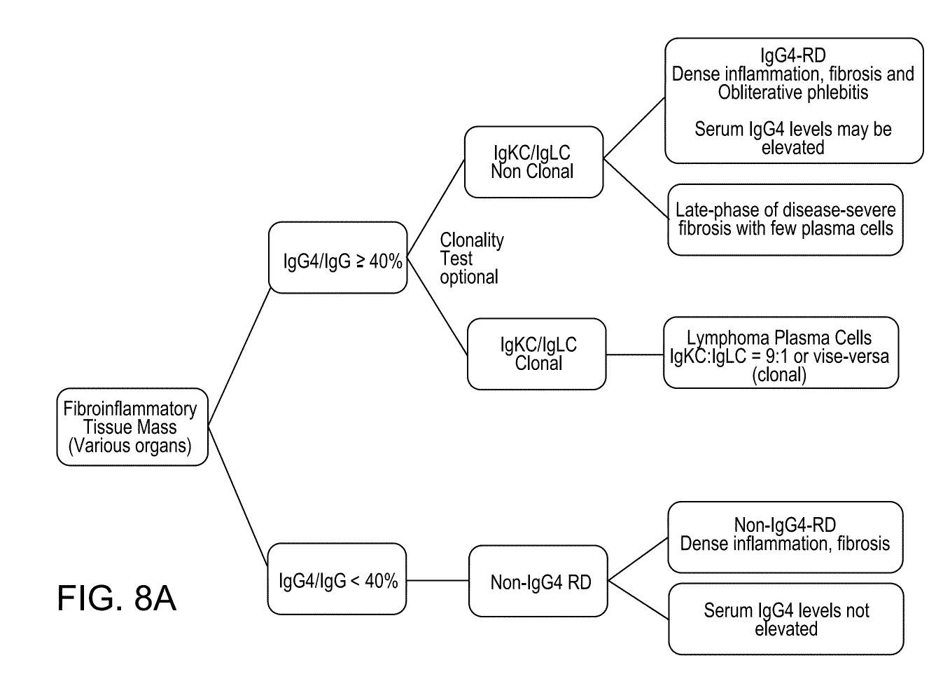

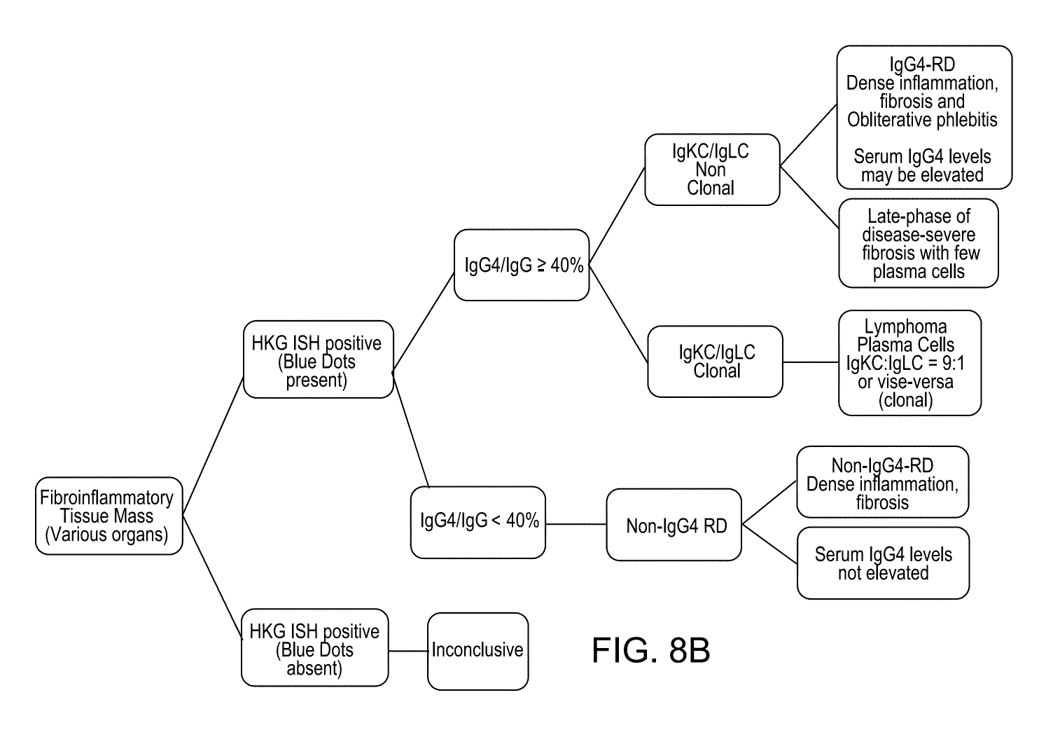

Once the numbers of IgG4+ and IgG+ cells in a sample are determined, as shown in FIGS. 8A-B, if the number of IgG4+ cells (preferably, the mean number in 3 HPF)/number of IgG+ cells (preferably, the mean number in 3 HPF) over a threshold, e.g., over 20%, 30%, 40%, or 50%, the sample is identified as likely being from a tumefactive lesion associated with an IgG4-RD. If the number of IgG4+ cells (preferably, the mean number in 3 HPF)/number of IgG+ cells (preferably, the mean number in 3 HPF) is below a threshold, e.g., below 20%, 30%, 40%, or 50%, the sample is identified as not likely to being from a tumefactive lesion associated with an IgG4-RD.

The detection of IgG+ and IgG4+ cells can be performed using methods known in the art; a preferred method is RNA in situ hybridization (RNA ISH). Other methods known in the art for gene expression analysis, e.g., RT-PCR, RNA-sequencing, and oligo hybridization assays including RNA expression microarrays, hybridization based digital barcode quantification assays such as the nCounter® System (NanoString Technologies, Inc., Seattle, Wash.), and lysate based hybridization assays utilizing branched DNA signal amplification such as the QuantiGene® 2.0 Single Plex and Multiplex Assays (Affymetrix, Inc., Santa Clara, Calif.); however, these non-RNA ISH methods cannot visualize RNA in situ, which is important in identifying the cell of origin and the retention of cellular morphology and other aspects that are lost when cells are lysed. Thus in some embodiments of the methods described herein RNA ISH methods are used wherein the cells are individually identifiable (i.e., although the cells are permeabilized to allow for influx and outflux of detection reagents, the structure of individual cells is maintained such that each cell can be identified); in contrast, methods such as RT-PCR, expression arrays, and so on use bulk samples wherein the RNA is extracted from disrupted cells, and the cells are not identifiable (and thus the cell of origin cannot be identified).

Certain RNA ISH platforms leverage the ability to amplify the signal within the assay via a branched-chain technique of multiple polynucleotides hybridized to one another (e.g., bDNA) to form a branch structure (e.g., branched nucleic acid signal amplification). In addition to its high sensitivity, the platform also has minimal non-specific background signal compared to immunohistochemistry. While RNA ISH has been used in the research laboratory for many decades, tissue based RNA diagnostics have only recently been introduced in the diagnostic laboratory. However, these have been restricted to highly expressed transcripts such as immunoglobulin light chains as low abundance transcripts such as IgG4 otherwise cannot be detected by a conventional RNA ISH platform (Hong et al., Surgery 146:250-257, 2009; Magro et al., J Cutan Pathol 30:504-511, 2003). This robust RNA ISH platform with its ability to detect low transcript numbers has the potential to revolutionize RNA diagnostics in paraffin tissue and other tissue assay sample formats.

In some embodiments, the assay is a bDNA assay, optionally a bDNA assay as described in U.S. Pat. Nos. 7,709,198; 7,803,541; 8,114,681 and 2006/0263769, which describe the general bDNA approach; see especially 14:39 through 15:19 of the '198 patent. In some embodiments, the methods include using a modified RNA in situ hybridization (ISH) technique using a branched-chain DNA assay to directly detect and evaluate the level of biomarker mRNA in the sample (see, e.g., Luo et al., U.S. Pat. No. 7,803,541B2, 2010; Canales et al., Nature Biotechnology 24(9):1115-1122 (2006); Ting et al., Aberrant Overexpression of Satellite Repeats in Pancreatic and Other Epithelial Cancers, Science 331(6017):593-6 (2011)). A kit for performing this assay is commercially-available from Affymetrix, Inc. (e.g., the QuantiGene® ViewRNA Assays for tissue and cell samples).

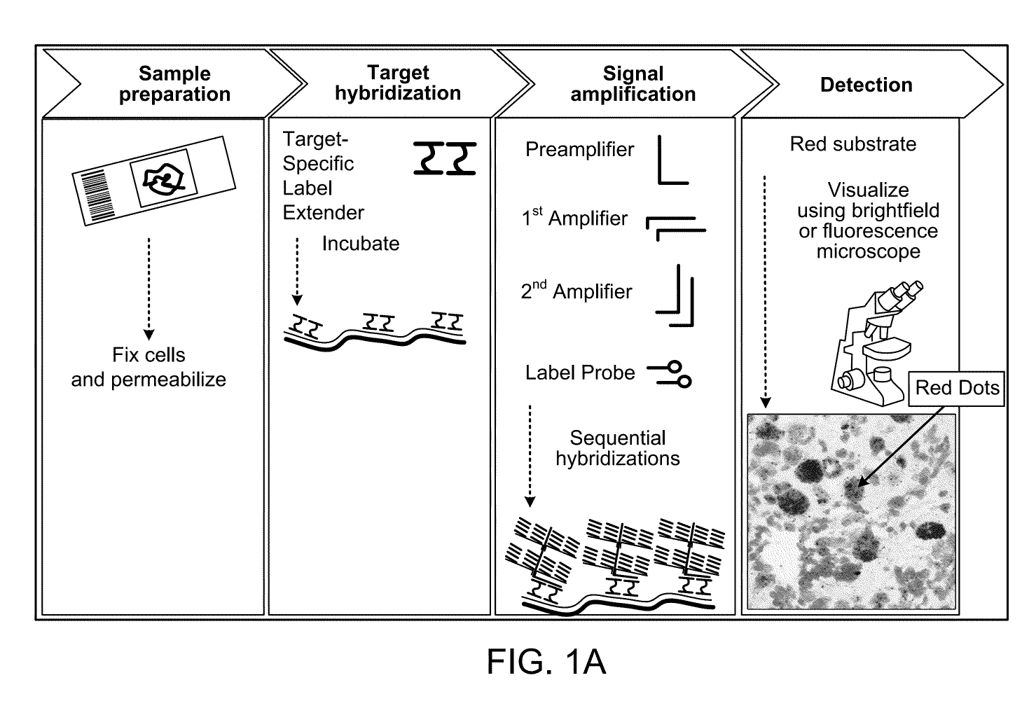

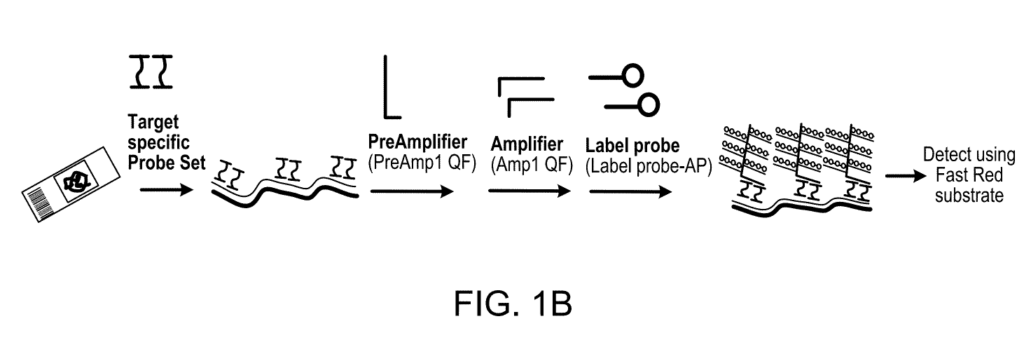

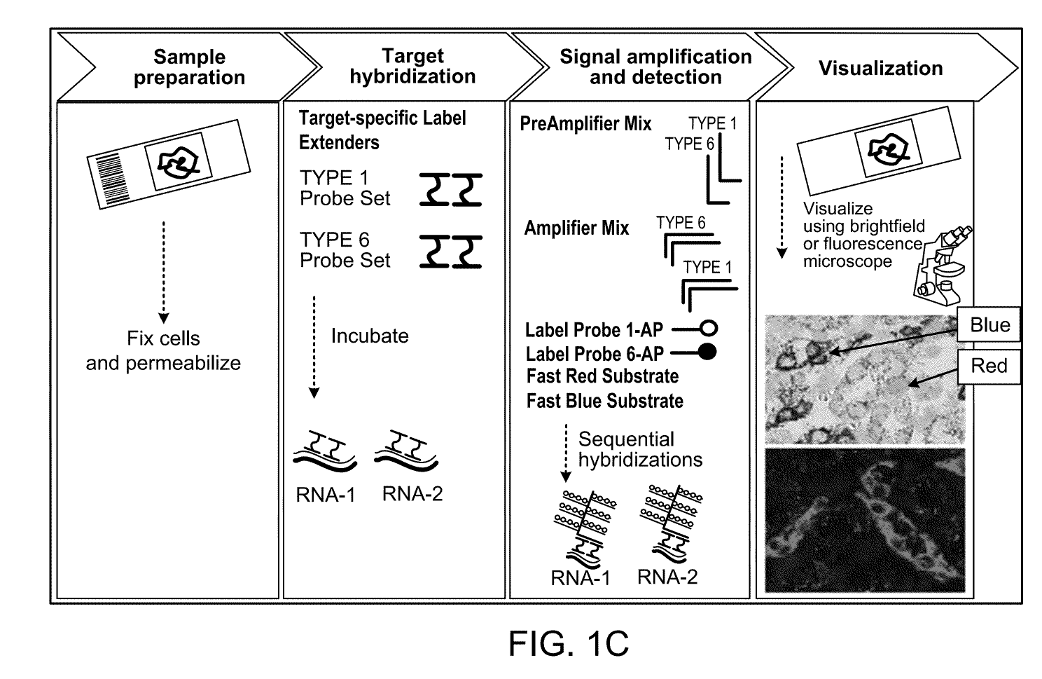

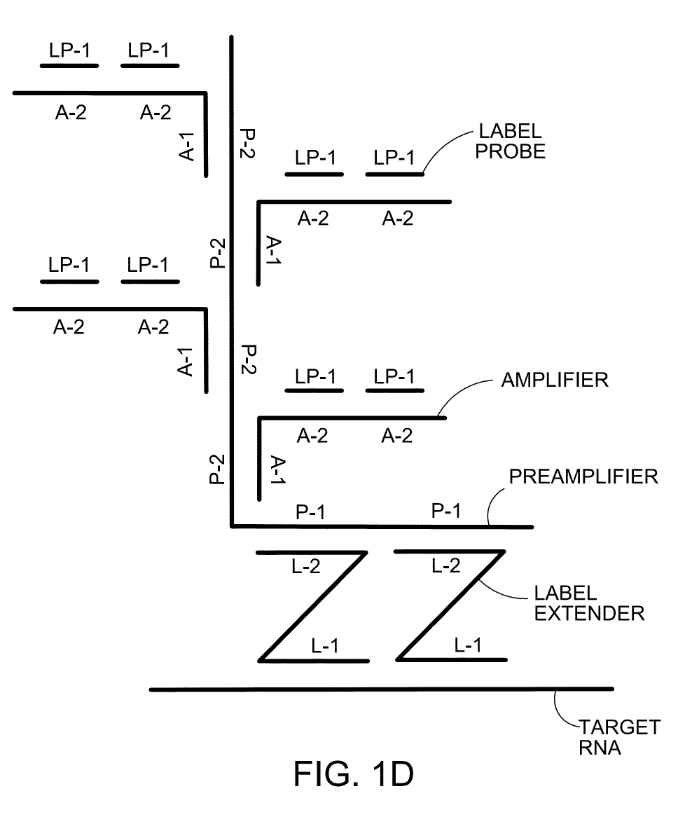

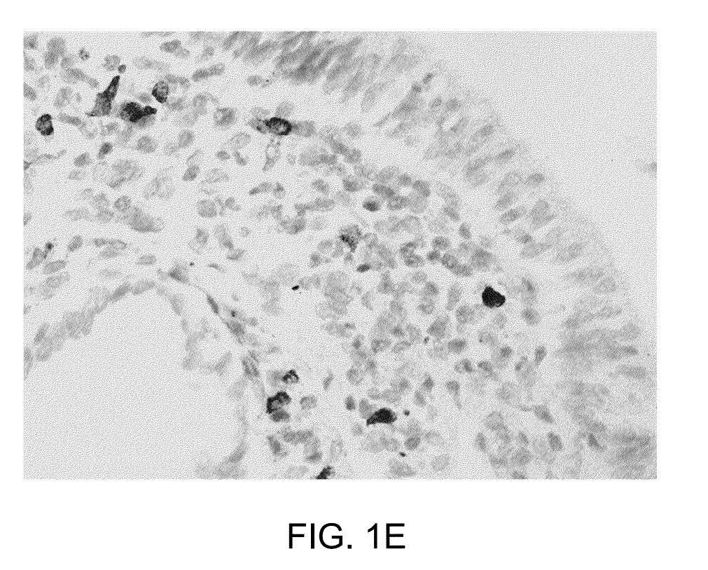

RNA ISH can be performed, e.g., using the ViewRNA™ technology (Affymetrix, Santa Clara, Calif.). ViewRNA ISH is based on the branched DNA technology wherein signal amplification is achieved via a series of sequential steps (e.g., as shown in FIGS. 1A-B in a single plex format and in FIG. 1C in a two plex format). Thus in some embodiments, the methods include performing an assay as described in US 2012/0052498 (which describes methods for detecting both a nucleic acid and a protein with bDNA signal amplification, comprising providing a sample comprising or suspected of comprising a target nucleic acid and a target protein; incubating at least two label extender probes each comprising a different L-1 sequence, an antibody specific for the target protein, and at least two label probe systems with the sample comprising or suspected of comprising the target nucleic acid and the target protein, wherein the antibody comprises a pre-amplifier probe, and wherein the at least two label probe systems each comprise a detectably different label; and detecting the detectably different labels in the sample); US 2012/0004132; US 2012/0003648 (which describes methods of amplifying a nucleic acid detection signal comprising hybridizing one or more label extender probes to a target nucleic acid; hybridizing a pre-amplifier to the one or more label extender probes; hybridizing one or more amplifiers to the pre-amplifier; hybridizing one or more label spoke probes to the one or more amplifiers; and hybridizing one or more label probes to the one or more label spoke probes); or US 2012/0172246 (which describes methods of detecting a target nucleic acid sequence, comprising providing a sample comprising or suspected of comprising a target nucleic acid sequence; incubating at least two label extender probes each comprising a different L-1 sequence, and a label probe system with the sample comprising or suspected of comprising the target nucleic acid sequence; and detecting whether the label probe system is associated with the sample). Each hybridized target specific polynucleotide probe acts in turn as a hybridization target for a pre-amplifier polynucleotide that in turn hybridizes with one or more amplifier polynucleotides. In some embodiments two or more target specific probes (label extenders) are hybridized to the target before the appropriate pre-amplifier polynucleotide is bound to the 2 label extenders, but in other embodiments a single label extender can also be used with a pre-amplifier. Thus, in some embodiments the methods include incubating one or more label extender probes with the sample. In some embodiments, the target specific probes (label extenders) are in a ZZ orientation, cruciform orientation, or other (e.g., mixed) orientation; see, e.g., FIGS. 10A and 10B of US 2012/0052498. Each amplifier molecule provides binding sites to multiple detectable label probe oligonucleotides, e.g., chromogen or fluorophore conjugated-polynucleotides, thereby creating a fully assembled signal amplification “tree” that has numerous binding sites for the label probe; the number of binding sites can vary depending on the tree structure and the labeling approach being used, e.g., from 16-64 binding sites up to 3000-4000 range. In some embodiments there are 300-5000 probe binding sites. The number of binding sites can be optimized to be large enough to provide a strong signal but small enough to avoid issues associated with overlarge structures, i.e., small enough to avoid steric effects and to fairly easily enter the fixed/permeabilized cells and be washed out of them if the target is not present, as larger trees will require larger components that may get stuck within pores of the cells (e.g., the pores created during permeabilization, the pores of the nucleus) despite subsequent washing steps and lead to noise generation. A simplified bDNA amplification scheme is shown in FIG. 1D.

In some embodiments, the label probe polynucleotides are conjugated to an enzyme capable of interacting with a suitable chromogen, e.g., alkaline phosphatase (AP) or horseradish peroxidase (HRP). Where an alkaline phosphatase (AP)-conjugated polynucleotide probe is used, following sequential addition of an appropriate substrate such as fast red or fast blue substrate, AP breaks down the substrate to form a precipitate that allows in-situ detection of the specific target RNA molecule. Alkaline phosphatase can be used with a number of substrates, e.g., fast red, fast blue, or 5-Bromo-4-chloro-3-indolyl-phosphate (BCIP). Thus in some embodiments, the methods include the use of alkaline phosphatase conjugated polynucleotide probes within a bDNA signal amplification approach, e.g., as described generally in U.S. Pat. No. 5,780,277 and U.S. Pat. No. 7,033,758. Other enzyme and chromogenic substrate pairs can also be used, e.g., horseradish peroxidase (HRP) and 3,3′-Diaminobenzidine (DAB). Many suitable enzymes and chromogen substrates are known in the art and can be used to provide a variety of colors for the detectable signals generated in the assay, and suitable selection of the enzyme(s) and substrates used can facilitate multiplexing of targets and labels within a single sample. For these embodiments, labeled probes can be detected using known imaging methods, e.g., bright-field microscopy (e.g., CISH).

Other embodiments include the use of fluorophore-conjugates probes, e.g., Alexa Fluor dyes (Life Technologies Corporation, Carlsbad, Calif.) conjugated to label probes. In these embodiments, labeled probes can be detected using known imaging methods, e.g., fluorescence microscopy (e.g., FISH). Selection of appropriate fluorophores can also facilitate multiplexing of targets and labels based upon, e.g., the emission spectra of the selected fluorophores.

In some embodiments, the assay is similar to those described in US 2012/0100540; US 2013/0023433; US 2013/0171621; US 2012/0071343; or US 2012/0214152. All of the foregoing are incorporated herein by reference in their entirety.

In some embodiments, an RNA ISH assay is performed without the use of bDNA, and the IgG and IgG4 specific probes are directly or indirectly (e.g., via an antibody) labeled with one or more labels as discussed herein.

The assay can be conducted manually or on an automated instrument, such the Leica BOND family of instruments, or the Ventana DISCOVERY ULTRA or DISCOVERY XT instruments.

In some embodiments, the detection methods use an RNA probe set targeting the human IgG or IgG4 mRNA transcripts, e.g., as shown in FIGS. 1A-C. The presence of a ratio of IgG4/IgG over a threshold, e.g., over 20%, 30%, 40%, or 50%, signals that the sample is likely to be from an IgG4-RD, while a ratio below that threshold indicates that it is not likely to be from an IgG4-RD; an exemplary decision tree is shown in FIG. 8A. As noted above, the levels of IgG and IgG4 can be determined in the same section, e.g., using a 2-plex assay with different labels, e.g., different chromogenic enzyme/substrate pairs (such as AP/fast red and HRP/DAB) (see FIG. 1C) or different fluorophores. Alternatively, the levels can be determined using a 1-plex assay in consecutive sections, e.g., using the same or different labels (see FIGS. 1A-B).

In some embodiments, the detection methods include detecting IgG and IgG4 in combination with pan-housekeeping (pan-HKG) genes, e.g. GAPDH, ACTB, or UBC, to assess RNA integrity, e.g., as shown in FIG. 1C. Cells that do not have expression of pan-HKG lack essential RNA integrity and hence need to be excluded from the analysis; an exemplary decision tree is shown in FIG. 8B. This eliminates false negative cases, as may arise with, e.g., improperly stored or prepared samples.

For example, in an embodiment wherein IgG and IgG4 are detected in consecutive sections, the 1^{st} tissue section can be used to detect IgG4 and HKG, and the 2^{nd} tissue section to detect IgG and HKG. In an embodiment wherein IgG and IgG4 are determined in the same section, IgG4, IgG and HKG are all determined in the same section, using three different labels. Both can be done in the same manner as the non-HKG tests, e.g., using chromogenic ISH (CISH) or fluorescence ISH (FISH). For CISH, one could use 3 different label probe systems, e.g., (1) alkaline phosphatase and fast red, (2) alkaline phosphatase and fast blue, and (3) horseradish peroxidase (HRP) and 3,3′-Diaminobenzidine (DAB). For FISH, an assay could employ 3 different fluorophores that have peak emissions with sufficient separation to allow distinct detection, such as peak emission values at, e.g., 519 nm, 665 nm, and 775 nm. Many suitable fluorophores are commercially available, e.g., Life Technologies offers Alexa Fluor dyes with peak emission values ranging from 442 nm to 814 nm, allowing straightforward fluorescent multiplexing.

Probes

Each probe set contains one or more, preferably multiple, polynucleotide probes (also referred to herein as label extenders for embodiments utilizing branched nucleic acid signal amplification). Each label extender probe consists of three parts with (1) part 1 designed to hybridize to the targeted gene, (2) part 2 being nucleotide spacer (e.g., 3-20 nucleotides) and (3) part 3 designed to hybridize to the unique tag within a bDNA preamplifier probe (see below and FIG. 1D).

| | | |
|---|---|---|
| Part1 (binds to target region) | Part2 (spacer) | Part3 (binds to bDNA) |

nnnnnnnnnnnnnnnnnnnnnnnnnSSSSSSSSSSSSSSBBBBBBBBBBBBBBBBBBB

The Part1 sequence of a probe can span a wide variety of lengths, from 12 bases to the full length of the target sequence, and will vary depending on the intended target and overall assay design characteristics (e.g., the desired hybridization temperature). Within certain embodiments, the Part1 sequence is preferably from 16 bases to 32 bases in length. The probe set for IgG can range from 1 or 2 polynucleotides to 26 polynucleotides or more, and the probe set for IgG4 can range from 1 or 2 polynucleotides to 8 polynucleotides or more, with the number of probes in each set depending on, e.g., the desired regions of each RNA target to be interrogated, the number of target regions desired in order to generate sufficient signal with the relevant detection approach of a particular assay, the contrast in total signal desired between IgG4 and IgG positive cells. In preferred embodiments, the Tₘ of each oligonucleotide is between 60° C. and 70° C.

The sequences of human IgG and IgG4 are known in the art. The IgG4 sequence is set forth in GenBank under Accession No. AJ294733, while the IgG sequence is set forth in GenBank under Accession No. GS00531); preferably, the IgG4 probe is isotype-specific while the IgG probe targets a conserved region.

In preferred embodiments, the probes that bind to IgG4 mRNA bind to a non-homologous constant region of Homo sapiens Ig heavy chain gamma4, when compared to other human immunoglobulin heavy chain constant regions e.g., gamma1, gamma2, gamma3, e.g., within the sequence

(SEQ ID NO: 1)

CAGCTTGGGCACGAAGACCTACACCTGCAACGTAGATCACAAGCCCAGCA
ACACCAAGGTGGACAAGAGAGTTGAGTCCAAATATGGTCCCCCATGCCCA
TCATGCCCAGCACCTGAGTTCCTGGGGGACCATCAGTCTTCCTGTTCCCC
CCAAAACC.

In preferred embodiments, the probes that bind to IgG mRNA bind to a conserved constant region of the four Homo sapiens Ig heavy gamma sequences, e.g., within the double-underlined portions of the following sequence:

(SEQ ID NO: 2)

GCAAGCTTCAAGGGCCCATCGGTCTTCCCCCTGGTGCCCTGCTCCAGGAG
CACCTCCGAGAGCACAGCCGCCCTGGGCTGCCTGGTCAAGGACTACTTCC
CCGAACCGGTGACGGTGTCGTGGAACTCATGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACGAAGACCTACACCTGCA
ACGTAGATCACAAGCCCAGCAACACCAAGGTGGACAAGAGAGTTGAGTCC
AAATATGGTCCCCCATGCCCATCATGCCCAGCACCTGAGTTCCTGGGGGG
ACCATCAGTCTTCCTGTTCCCCCCAAAACCCAAGGACACTCTCATGATCT
CCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCA
GGGTCCTCACCGTCCTGCACCAGGACTGGCTGAACGGTAAGGAGTACAAG
TGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCAT
CCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCC
GGAGGACAACTACAAGACCACGCCTCCCGTGCTGGACTCCGACGGCTCCT
TCTTCCTCTACAGCAGGCTAACCGTGGACAAGAGCAGGTGGCAGGAGGGG
AATGTCTTCTCATGCTCCGTGATGCATGAGGCTCTGCACAACCACTACAC
ACAGAAGAGCCTCTCCCTGTCTCCGGGTAAA

Exemplary probes are shown in Table 1C within Example 3, below. In some embodiments, the one or more polynucleotide probes that bind specifically to IgG4 mRNA are selected from the IgG4 probes in Table 1C. Additionally or alternatively, the one or more polynucleotide probes that bind specifically to IgG mRNA are selected from the IgG probes in Table 1C.

One of skill in the art would readily be able to identify sequences for additional species bioinformatically, and would appreciate that the sequence of IgG and IgG4 mRNA used should match the species of the subject from which the sample is obtained. The subject is preferably a mammal and can be, e.g., a human or veterinary subject (e.g., cat, dog, horse, cow, or sheep).

Ruling Out Lymphoma

An IgG4/IgG ratio over the threshold is a powerful indicator that an IgG4-RD is at issue, as opposed to a non-IgG4-RD. However, even with a qualifying ratio, it is possible that an IgG4 related lymphoma is at issue, thus leading to a potential differential diagnosis situation. With reference to the diagnostic flowcharts in FIGS. 8A-B, the initial determination (outside of possible housekeeping gene use to, e.g., assess RNA integrity) is whether the IgG4/IgG ratio is over a threshold (e.g., over 20%, 30%, 40%, or 50%). However, even with a ratio over the utilized threshold, there is still a possibility that the individual at issue has an IgG4 related lymphoma as opposed to an IgG4-RD. Thus, in some embodiments, the methods include making a differential diagnosis of IgG4-RD versus an IgG4 lymphoma, which can also be used to help guide treatment of a patient. These embodiments can include making a determination of the clonal/non-clonal aspect of the mass, e.g., the clonality of the cells that are present, which can be, e.g., plasma cells, lymphocytes. This determination can be performed using, e.g., RNA ISH for IgKC and IgLC, or through other routes, e.g., RT-PCR, which is presently the more prevalent method as immunohistochemistry approaches suffer from low signal to background noise ratios. See, e.g., Morrison and Caliguiri, Diagnostic Molecular Pathology 10(3):171-178 (2001); Van Dongen et al., Leukemia. 17(12):2257-317 (2003). Therefore, the measurement of the expression of the kappa and lambda light chain RNA can serve as a confirmation that one is truly dealing with an IgG4 RD and not an IgG4 lymphoma. If IgG4 plasma cells show IgKC/IgLC clonality, as evidenced by, e.g., a high ratio of IgKC:IgLC expression (or vice-versa) in comparison to the normal ratios of, e.g., 2-3:1 (or vice-versa) (see, e.g., Rizzo and Nassiri, “Diagnostic Workup of Small B Cell Lymphomas: A Laboratory Perspective,” Lymphoma, vol. 2012, Article ID 346084, 15 pages, 2012) then a diagnosis of IgG4 related lymphoma is to be considered. In some embodiments, the IgKC and IgLC expression is measured by using an additional section of the tissue mass at issue. In other embodiments, such as but not limited to FISH embodiments with a sufficient number of distinct fluorophores, the IgKC and IgLC expression can be measured at the same time as the IgG4 and IgG expression (which may also be accompanied by measurement of a selected housekeeping gene as well).

Treatment

While neoplasms may be treated with surgical excision with or without adjuvant therapy, IgG4-RD is usually treated with immuno-suppressants such as steroids. In some instances, Azathioprine, Methotrexate, and/or Rituximab (B-cell depleting agent) may be used as treatment options. In some cases where disease involvement is not extensive or affects a non-vital organ, no treatment is required. Thus the methods described herein can include selecting and administering a treatment for a subject who has been identified as having an IgG4-RD, plasma cell lymphoma, or a non-IgG4-RD, e.g., a neoplastic tumor. Where the subject is determined to have a neoplastic tumor, the tissue of origin can be determined (e.g., primary versus metastatic) and an appropriate treatment administered (see, e.g., the NCCN cancer treatment guidelines; ASCO treatment guidelines; ESMO treatment guidelines; Oxford Textbook of Oncology, Second Edition; Textbook of Medical Oncology, Informa Healthcare; Comprehensive Textbook of Oncology).

Kits

There are provided herein kits comprising reagents for performing any of the methods described herein. In some embodiments, a kit comprises one or more polynucleotide probes that are capable of binding specifically to IgG4 mRNA in situ and one or more polynucleotide probes that are capable of binding specifically to IgG mRNA in situ.

In some embodiments, a kit comprises one or more label extender probes that are capable of binding to one or more target regions in the IgG4 mRNA and one or more label extender probes that are capable of binding to one or more target regions in the IgG mRNA.

In some embodiments the one or more polynucleotide probes that are capable of binding specifically to IgG4 mRNA in situ comprise one or more label extender probes that are capable of binding to one or more target regions in the IgG4 mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

In some embodiments the one or more polynucleotide probes that are capable of binding specifically to IgG mRNA in situ comprise one or more label extender probes that are capable of binding to one or more target regions in the IgG mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

In some embodiments the kit further comprises one or more polynucleotide probes that bind specifically to IgKC mRNA in situ and/or one or more polynucleotide probes that bind specifically to IgLC mRNA in situ.

In some embodiments, the kit comprises one or more label extender probes that are capable of binding to one or more target regions in the IgKC mRNA and one or more label extender probes that are capable of binding to one or more target regions in the IgLC mRNA.

In some embodiments, the one or more polynucleotide probes that are capable of binding specifically to IgKC mRNA in situ comprise one or more label extender probes that are capable of binding to one or more target regions in the IgKC mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes. Additionally or alternatively, the one or more polynucleotide probes that are capable of binding specifically to IgLC mRNA in situ comprise one or more label extender probes that are capable of binding to one or more target regions in the IgLC mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes

In some embodiments the kit further comprises one or more polynucleotide probes that bind specifically to mRNA encoding a housekeeping gene (HKG) in situ. In some embodiments, the kit comprises one or more label extender probes that are capable of binding to one or more target regions in the HKG mRNA

In some embodiments, the one or more polynucleotide probes that are capable of binding specifically to mRNA encoding a HKG in situ comprise one or more label extender probes that are capable of binding to one or more target regions in the HKG mRNA, one or more pre-amplifier probes that are capable of hybridizing to the one or more label extender probes, one or more amplifier probes that are capable of hybridizing to the one or more pre-amplifier probes, and one or more label probes that are capable of hybridizing to the one or more amplifier probes.

### EXAMPLES

The invention is further described in the following examples, which do not limit the scope of the invention described in the claims.

Statistical Analysis

Statistics were calculated using SPSS version 21.0 (SPSS, Chicago, Ill., USA). Differences between groups were evaluated using the Student t-test for quantitative variables. A P-value <0.05 was considered significant.

### Example 1

### IgG4-Related Disease Patients and IgG4-Related Disease Mimickers Cohort

53 cases were evaluated in total. These cases included biopsies from 22 subjects with IgG4-related disease. The mean age of the 22 subjects with IgG4 related disease was 60 years (range 41 to 85). Fifteen of these subjects were male and 7 were female. The sites/organs involved by the disease are listed in Table 1A.

TABLE 1A
Site of disease for the IgG4-Related disease and control groups

| | IgG4 related | Mimic of IgG4 |
|---|---|---|
| Organ | disease | related disease |
| Ampulla/Pancreas | 3 | 0 |
| Liver | 1 | 1 |
| Pancreas | 7 | 4 |
| Lung | 2 | 7 |
| Lymph node | 1 | 3 |
| Retroperitoneal and | 1 | 6 |
| mesentery | | |
| Orbit | 0 | 2 |
| Pachymeningitis | 0 | 1 |
| Pituitary | 0 | 2 |
| Pleura | 1 | 0 |
| Salivary gland | 3 | 5 |
| Oropharynx | 3 | 0 |
| Total | 22 | 31 |

The IgG4-related disease mimickers cohort, identified both prospectively and retrospectively, was composed of 31 subjects with disorders that mimic IgG4-related disease in their clinical, serological, or histopathological presentations (Table 1B). The mean age of this group was 57 years (range 24 to 84) (P=0.4 for comparison to IgG4-related disease group) and the group was comprised of 16 males and 15 females.

TABLE 1B
Final diagnosis in the non-IgG4 related disease series

| Diagnosis | Number of cases |
|---|---|
| Wegener's granulomatosis | 6 |
| Chronic sialadenitis - not otherwise specified | 3 |
| Lymphoepithelial sialadenitis | 2 |
| Malignancy (pancreas, lung) | 3 |
| Retroperitoneal fibrosis | 5 |
| Rheumatoid arthritis | 2 |
| Reactive lymph nodes | 3 |
| Chronic pancreatitis | 2 |
| Hypophysitis | 2 |
| Primary sclerosing cholangitis, non-specific interstitial | 1 each |
| pneumonia, orbital pseudotumor | |

Subjects in both cohorts encompassed a broad range of organ involvement (Table 1A). Cases prior to 2004 were collected retrospectively; subsequent cases were identified in a prospective database.

The criteria used to establish a diagnosis of IgG4-related disease were based on a recently published consensus document.⁵ The diagnosis of IgG4-related disease required the presence of one or more of these histologic features: 1) a dense lymphoplasmacytic infiltrate; 2) storiform-type fibrosis; and, 3) obliterative phlebitis, as well as elevated numbers of IgG4 positive plasma cells. The appearance on imaging, serum IgG4 levels, the presence of multiorgan involvement compatible with IgG4 related disease and favorable response to glucocorticoids was also factored into the clinical diagnosis.

### Example 2

### Serum IgG4

Information on serum IgG4 concentrations was available for 11 IgG4 related disease cases (50%) and 5 cases (16%) in the control arm. The mean serum IgG4 concentration in the IgG4 related disease cohort was 306 milligrams/deciliter (range 67-779), while that of the non-IgG4 related disease arm was 72.3 milligrams/deciliter (range 9-125)(P=0.07). Four of the 9 IgG4 related disease cases had serum IgG4 concentrations >140 milligrams/deciliter, but none of those in the mimickers group had serum IgG4 concentration elevations of that magnitude.

### Example 3

### Validation of the IgG4 In Situ Hybridization Platform

In situ hybridization was performed using the ViewRNA™ technology (Affymetrix, Santa Clara, Calif.). ViewRNA in situ hybridization is based on the branched DNA technology wherein signal amplification is achieved via a series of sequential steps. Each pair of bound target probe set oligonucleotides acts a template to hybridize a pre-amplifier molecule that in turn binds multiple amplifier molecules. Each amplifier molecule provides binding sites to multiple alkaline phosphatase (AP)-conjugated-oligonucleotides thereby creating a fully assembled signal amplification “tree” that has approximately 400 binding sites for the AP-labeled probe. Following sequential addition of the fast-red substrate, AP breaks down the substrate to form a precipitate (red dots) that allows in-situ detection of the specific target RNA molecule (FIG. 1E).

In situ hybridization probes (Affymetrix, Santa Clara, Calif.) were designed against the IgG4 and IgG transcripts as identified in the NCBI nucleotide database. The IgG4 probe is isotype-specific (and targeted the sequence set forth in GenBank under Accession No. AJ294733), while the IgG probe targets RNA sequences to all subclasses of IgG (the sequence set forth in GenBank under Accession No. GS00531); the sequences of the target specific probes (or at least the portion of the probes that are intended to hybridize with the target RNA) are set forth in Table 1C.

TABLE 1C
IgG probes

| | | | | SEQ |
|---|---|---|---|---|
| | | | | ID |
| Position | oligo sequences | Tm | Length | NO: |
| 70-86bp | accaggcagcccagggc | 66.9 | 17 | 3 |
| 87-107bp | ggttcggggaagtagtccttg | 64.5 | 21 | 4 |
| 108-128bp | gagttccacgacaccgtcacc | 65.9 | 21 | 5 |
| 129-146bp | ccgctggtcagggcgcat | 70.7 | 18 | 6 |
| 147-163bp | ccgggaaggtgtgcacg | 63.8 | 17 | 7 |
| 164-187bp | agagtcctgaggactgtaggacag | 62.8 | 24 | 8 |
| 188-206bp | accacgctgctgagggagt | 63.5 | 19 | 9 |
| 207-223bp | tgctggagggcacggtc | 63.2 | 17 | 10 |
| 461-483bp | gccatccacgtaccagttgaact | 67.2 | 23 | 11 |
| 484-505bp | tcttggcattatgcacctccac | 66.5 | 22 | 12 |
| 632-654bp | tttggagatggttttctcgatgg | 67.5 | 23 | 13 |
| 655-671bp | cggggctgccctttggc | 70.3 | 17 | 14 |
| 672-693bp | cagggtgtacacctgtggctct | 65.3 | 22 | 15 |
| 695-714bp | catctcctcctgggatgggg | 67.3 | 20 | 16 |
| 715-736bp | tcaggctgacctggttcttggt | 67.4 | 22 | 17 |
| 737-756bp | gaagcctttgaccaggcagg | 65.9 | 20 | 18 |
| 757-774bp | ggcgatgtcgctggggta | 65.6 | 18 | 19 |
| 775-795bp | cccattgctctcccactccac | 67.7 | 21 | 20 |
| 796-817bp | tcttgtagttgtcctccggctg | 66 | 22 | 21 |
| 818-834bp | cagcacgggaggcgtgg | 66.7 | 17 | 22 |
| 835-855bp | gaagaaggagccgtcggagtc | 66.7 | 21 | 23 |
| 856-877bp | ccacggttagcctgctgtagag | 66 | 22 | 24 |
| 878-898bp | cctcctgccacctgctcttgt | 67.8 | 21 | 25 |
| 899-921bp | cacggagcatgagaagacattcc | 67.6 | 23 | 26 |
| 922-942bp | gttgtgcagagcctcatgcat | 64.6 | 21 | 27 |
| 943-967bp | gggagaggctcttctgtgtgt | 68 | 25 | 28 |
| | agtg | | | |

IgG4 probes

| | | | | SEQ |
|---|---|---|---|---|
| | | | | ID |
| Position | oligo sequence | Tm | length | NO: |
| 66-89bp | Gaccatatttggactcaactctct | 60.9 | 24 | 29 |
| 90-107bp | Ggcatgatgggcatgggg | 67.91 | 8 | 30 |
| 110-127bp | Ccccaggaactcaggtgc | 61.2 | 18 | 31 |
| 128-149bp | Ggaacaggaagactgatggtcc | 63.9 | 22 | 32 |

These probe sets were used in conjunction with the ViewRNA Tissue Assay Kit (2-plex) and in situ hybridization was performed according to the manufacturer's instructions. Briefly, dissected tissues were fixed for <24 hours in 10% Neutral Buffer Formalin at room temperature, followed by the standard formaldehyde-fixed, paraffin-embedded (FFPE) preparation. The FFPE tissues were sectioned at 5+/−1 micron and mounted on Surgipath X-tra glass slide (Leica BioSystems, Buffalo Grove, Ill.), baked for 1 hour at 60° C. to ensure tissue attachment to the glass slides, and then subjected to xylene deparaffinization and ethanol dehydration. To unmask the RNA targets, dewaxed sections were incubated in 500 ml pretreatment buffer (Affymetrix/Santa Clara, Calif.) at 90-95° C. for 10 minutes and digested with 1:100 dilution protease at 40° C. (Affymetrix, Santa Clara, Calif.) for 10 minutes, followed by fixation with 10% formaldehyde at room temperature for 5 minutes. Unmasked tissue sections were subsequently hybridized with 1:40 dilution IgG4 or IgG probe sets for 2 hours at 40° C., followed by series of post-hybridization washes. Signal amplification was achieved via a series of sequential hybridizations and washes as described in the user's manual. Slides were post-fixed with 4% formaldehyde, counterstained with Gill's hematoxylin, mounted using Advantage Mounting Media (Innovex, Richmond, Calif.), and visualized using a standard bright-field microscope. An attempt was made to identify the same three HPFs that were examined on an immunohistochemical platform, and quantification was performed on similar lines.

Immunohistochemistry for IgG4 and IgG was also performed as described previously.^{9, 10} In brief, immunohistochemical studies using antibodies to IgG4 (Zymed, 1:200 dilution) and IgG (Dako, 1; 3000) were performed. Antigen retrieval was conducted after protease digestion, and antigen detection was achieved using UltraView diaminobenzidine chromogen (Ventana Medical Systems; Tucson, Ariz.). Three high power fields with the highest number of IgG4-positive cells were identified and the mean counts in these fields were recorded. The number of IgG-positive plasma cells within these 3 fields was also recorded, enabling the derivation of IgG4 to IgG ratio.

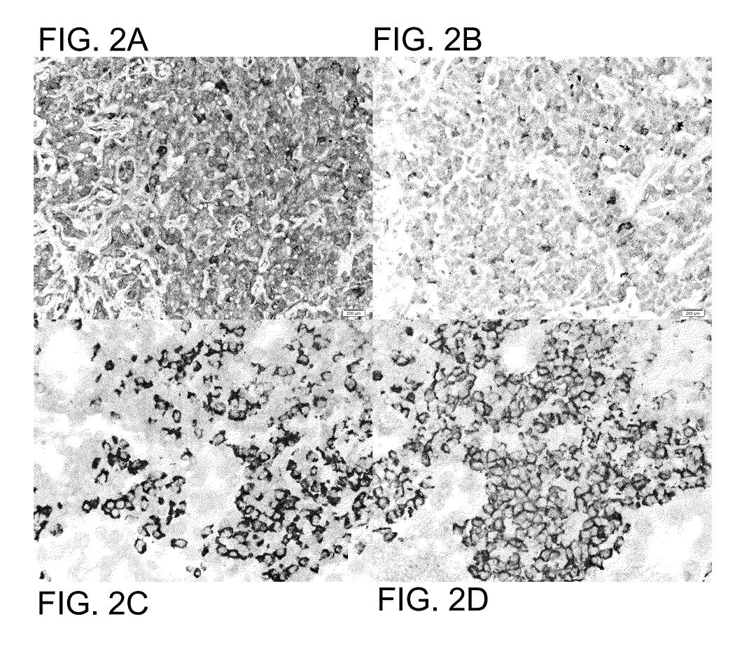

In order to validate the RNA-ISH platform, ISH results were compared to the currently-accepted gold standard immunohistochemistry for IgG4 (FIGS. 2A-D, 3A-B, 4A-D). 19 cases in which enumeration of IgG4 or IgG by IHC could not be performed were not included in this analysis. In all of the remaining 24 cases, both RNA-ISH and IHC produced concordant results, with the same assignment of patients to the IgG4 related disease category. There was no significant difference between the mean IgG4 counts performed on the 2 platforms (P=0.17). However, the mean number of IgG-positive plasma cells per high power field on in situ hybridization was higher than immunohistochemistry (P=0.006). Accordingly, the IgG4:IgG ratio on the in situ hybridization platform was significantly lower (P=0.03) compared with the estimate derived from immunohistochemistry.

Both the immunohistochemical platform as well as in situ hybridization identified higher numbers of IgG4 positive plasma cells and a higher IgG4 to IgG ratio in patients with IgG4 related disease (see Table 3, below). However, IgG4 in situ hybridization provided a more robust separation between IgG4-related disease and mimickers of IgG4-related disease (FIGS. 2A-D). The IgG4 to IgG ratio performed on the in situ hybridization platform was also more effective in distinguishing IgG4-related disease from cases that mimicked this condition (FIGS. 2A-D).

### Example 4

### IgG4-Related Disease Cases with Suboptimal Performance on the Immunoperoxidase Platform

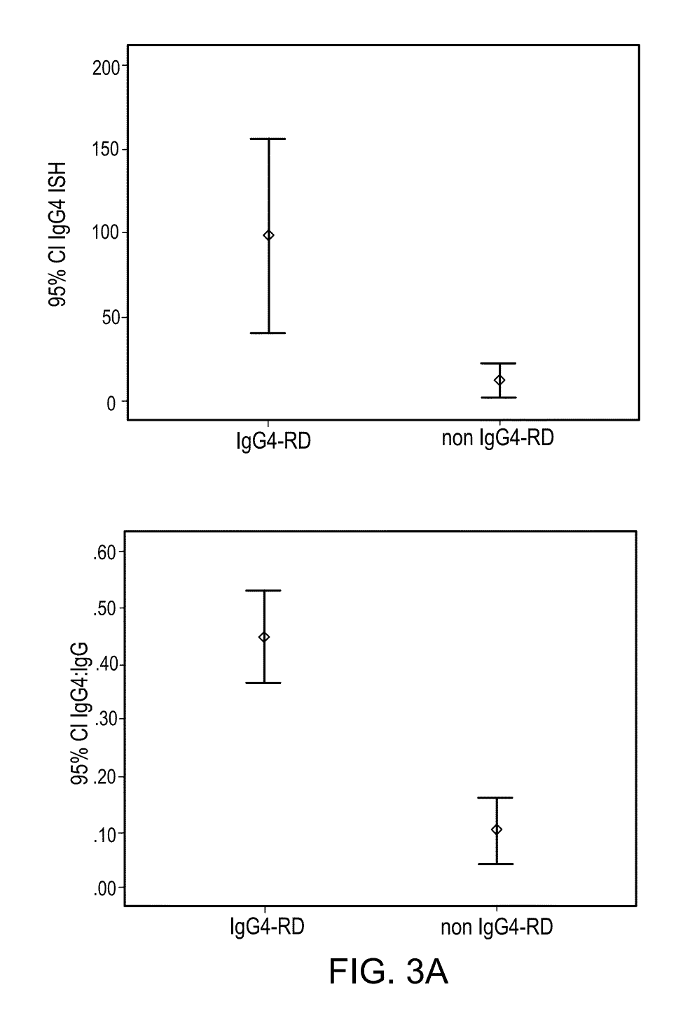

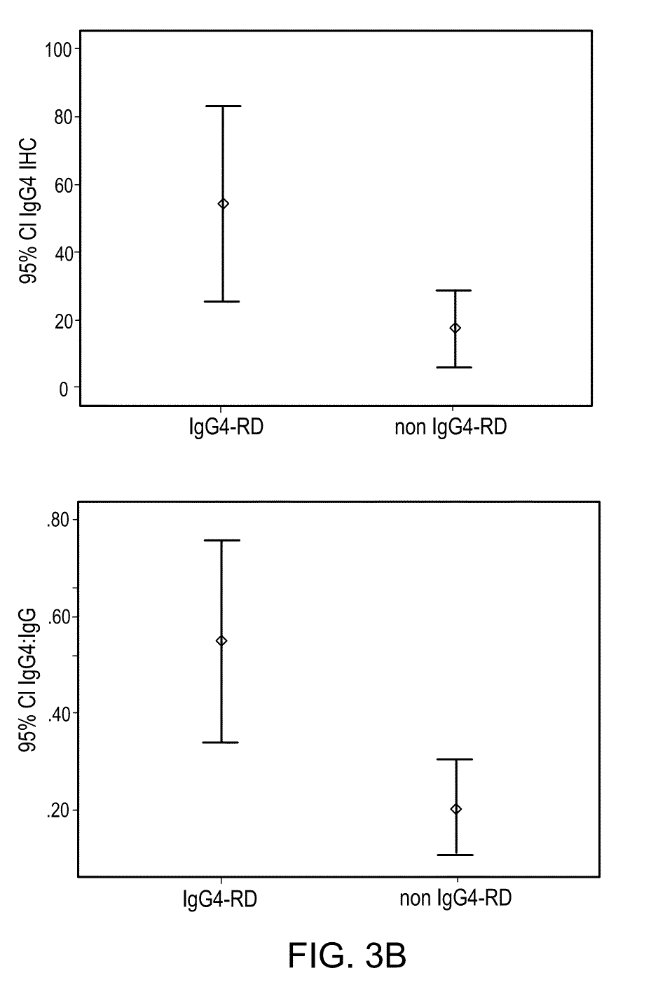

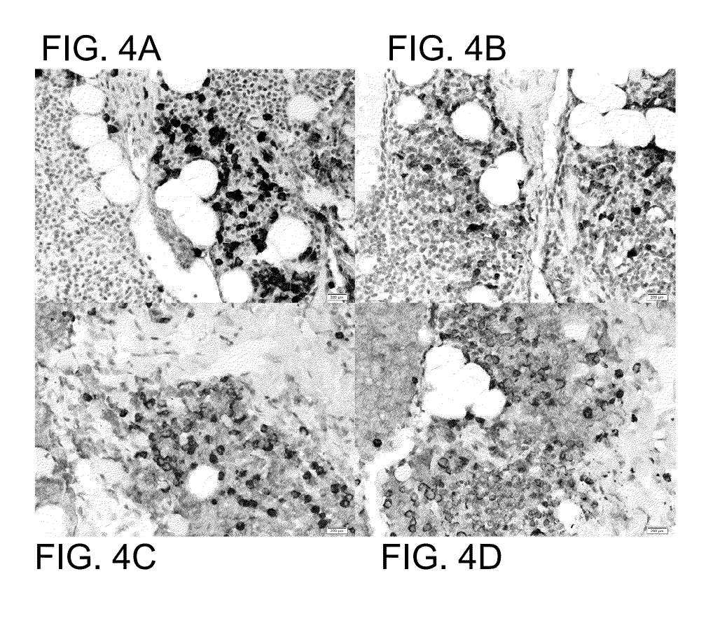

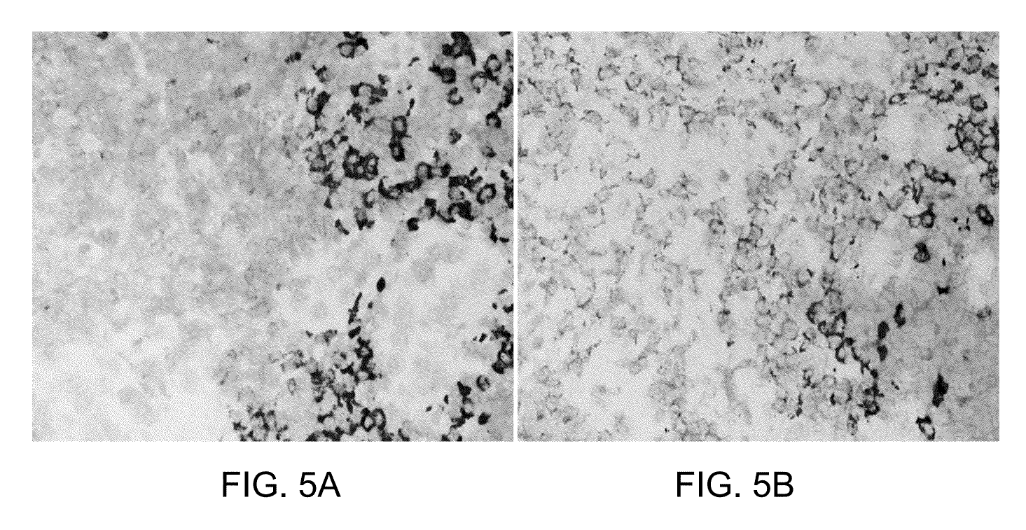

In seven IgG4-related disease cases (32%) (Table 2), the inability to enumerate either or both the IgG4- or IgG-positive cells by immunohistochemistry within tissue samples compromised the eventual histopathologic diagnosis, including 4 biopsy samples and a single pulmonary resection (FIGS. 2A-D & 3A-B). In contrast to immunohistochemistry, the signal on the in situ hybridization platform was confined to lymphocytes and plasma cells, resulting in essentially no background staining (FIGS. 3A-B). The in situ hybridization stains facilitated the enumeration of IgG4 and IgG positive cells and thus validated the diagnosis of IgG4 related disease in the ampullary, pancreatic, and oropharyngeal biopsies. In addition, one of these biopsies showed large numbers of IgG4-positive lymphocytes.

TABLE 2
IgG4 related disease: cases in which the in situ hybridization outperformed immunohistochemistry

| | | | | | IgG4 | | |
|---|---|---|---|---|---|---|---|
| | Site of | | In situ | Immuno- | positive | | |
| | biopsy/other | Histological | hybridization | histochemistry | lymphocytes | | |
| | sites of | features of | IgG4/IgG | IgG4/IgG | on in situ | Serum | Response to |
| | disease | IgG4-RD | Per HPF | Per HPF | hybridization | IgG4 | therapy |
| 1 | Ampulla/ | Dense LP | 15/80 | 0/could not be | No | 640 | Responded to |
| | pancreas | infiltrate | | interpreted | | | steroids |
| 2 | Ampulla/ | Dense LP | 5/10 | 8/could not be | No | 102 | Biliary and |
| | systemic | infiltrate | | interpreted | | | pulmonary |
| | disease | | | | | | manifestations |
| | involving | | | | | | responded to |
| | orbit & lung & | | | | | | steroids |
| | thyroid | | | | | | |
| 3 | Pharyngeal | Dense LP | 75/210 | Could not be | No | 196 | Responded to |
| | and | infiltrate | | interpreted | | | rituximab |
| | laryngeal | | | | | | |
| | mass | | | | | | |
| 4 | Salivary | Lymphocytes | 1/10 | Could not be | Present | 240 | Responded to |
| | gland-fine | only | | interpreted | | | rituximab |
| | needle | | | | | | |
| | aspiration | | | | | | |
| | biopsy | | | | | | |
| 5 | Lung | Dense LP | 200/350 | Could not be | Present | NA | No therapy |
| | | infiltrate + | | interpreted | | | |
| | | storiform | | | | | |
| | | fibrosis | | | | | |
| 6 | Pancreas | Storiform | 21/44 | Could not be | No | 240 | Responded to |
| | | type fibrosis, | | interpreted | | | steroids |
| | | lymphocytes | | | | | |
| | | and plasma | | | | | |
| | | cells | | | | | |
| 7 | Pancreas | Fibrosis, | 12/28 | Could not be | No | 177 | Responded to |
| | | lymphocytes | | interpreted | | | steroids |
| | | and plasma | | | | | |
| | | cells | | | | | |

IgG4-RD: IgG4 related disease
LP: Lymphoplasmacytic

In addition to these seven cases, the immunohistochemical preparations for IgG could not be quantified in four cases. However, the morphological features in conjunction with the immunoperoxidase stain for IgG4 permitted a histological diagnosis of IgG4-RD. A fine needle aspiration from a submandibular salivary gland swelling yielded only a few lymphocytes. The in situ hybridization stain failed to identify IgG4-positive plasma cells, but occasional IgG4-positive lymphocytes were identified. Therapy with rituximab was initiated, based primarily on a clinical suspicion, with complete resolution of the submandibular salivary gland swelling.

### Example 5

### Non-IgG4 Related Disease Cases with Suboptimal Performance of the Immunoperoxidase Platform

In six cases of IgG4-related mimickers, the immunohistochemical stain for IgG showed high levels of nonspecific stain, precluding quantitative analysis. The IgG4 and IgG in situ hybridization stains showed a signal within plasma cells that was of sufficient clarity to classify these cases appropriately.

### Example 6

### IgG4 Count and Ratio: IgG4-Related Disease Cases Versus Mimickers

Both the immunohistochemical platform as well as in situ hybridization identified higher numbers of IgG4 positive plasma cells and a higher IgG4 to IgG ratio in patients with IgG4 related disease (see Table 3). However, IgG4 in situ hybridization provided a more robust separation between IgG4-related disease and mimickers of IgG4-related disease (FIGS. 3A-B). The IgG4 to IgG ratio performed on the in situ hybridization platform was also more effective in distinguishing IgG4-related disease from cases that mimicked this condition (FIGS. 3A-B).

TABLE 3
IgG4 counts and IgG4:IgG ratio on in situ hybridization
and immunohistochemical platforms

| | | Mimics of | |
|---|---|---|---|
| | IgG4 related | IgG4 related | |
| | disease | disease | P value |
| IgG4 ISH | 98.5 (122.8) | 12.1 (24.9) | 0.0001 |
| IgG4/IgG ISH | 0.45 (0.17) | 0.1 (0.15) | 0.0001 |
| IgG4 IHC | 95 (117) | 15 (27) | 0.015 |
| IgG4/IgG ISH | 0.54 (0.23) | 0.18 (0.23) | 0.0001 |

### Example 7

### In Situ Hybridization Signal within Lymphocytes

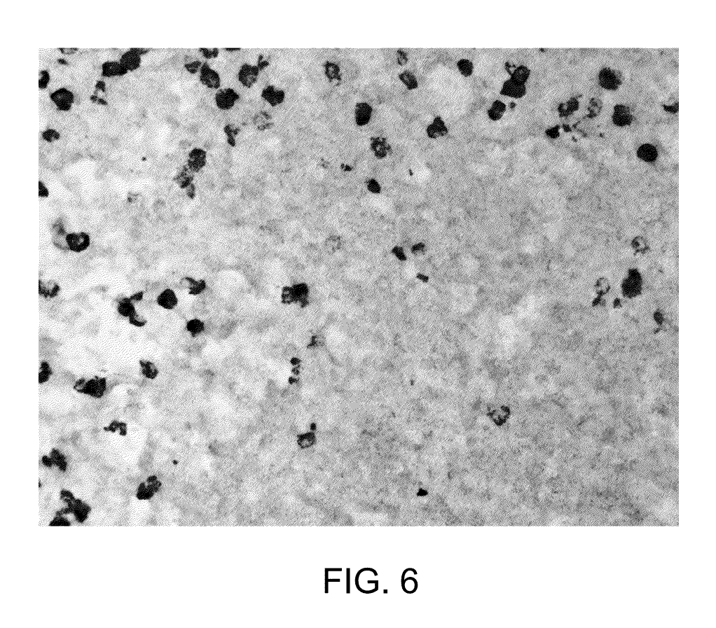

The stains for both IgG and IgG4 showed a strong signal within plasma cells and there was no background stain (FIGS. 5-6). However, in a subset of cases, a weaker signal was also detected in lymphocytes (FIG. 6). The cytoplasmic signal in lymphocytes was best appreciated in biopsies performed after 2008, but biopsies as old as 10 years showed positive signal in lymphocytes.

No positive IgG4 signal was identified in lymphocytes that lacked an IgG signal. Positive IgG signal was identified in 41 cases from the cohort overall, 15 from the IgG4-RD cohort and 26 from the non-IgG4-related disease mimicker cases. Biopsies from 11 of the 15 patients (73%) in the IgG4-related cohort showed IgG4 reactivity within lymphocytes. A sheet-like pattern of reactivity was seen in 4 of these cases (FIG. 6). Biopsies from 4 of the 26 cases (15%) in the non-IgG4 cohort showed positive signal in the cytoplasm of lymphocytes. The 4 cases of non-IgG4-related disease that showed IgG4+ lymphocytes included 3 lung biopsies from subjects with granulomatosis with polyangiitis (formerly Wegener's granulomatosis) and 1 from a subject with rheumatoid pachymeningitis. However, only occasional IgG4-positive lymphocytes were detected in those cases. No sheet-like patterns of IgG4 reactivity was observed in any of the biopsies from subjects in the IgG4-related disease mimickers cohort.

Other investigators have also noted difficulties in counting IgG-bearing plasma cells on immunohistochemistry, and it has been observed that the immunohistochemical platform occasionally yields an IgG4 to IgG ratio of greater than 1, particularly when the number of IgG4 positive plasma cells is high.⁴ In comparison to the in situ hybridization stain, the IgG immunohistochemical signal tends to be less bright and to show significant, often confounding background signal.

The in situ hybridization platform proved superior to immunohistochemistry, even in instances where enumeration of IgG4 and IgG bearing cells could be performed on both platforms. In particular, the in situ hybridization platform was superior to immunohistochemistry in separating the two patient cohorts on the basis of the IgG4:IgG ratio. Based on the cases examined for the purposes of this study, a cutoff value for the IgG4 to IgG ratio as measured through the in situ hybridization platform may be somewhat lower than that recommended for conventional immunohistochemistry technique (30%).⁵

Reactivity of lymphocytes on IgG staining is observed occasionally on immunohistochemistry studies, but lymphocyte reactivity for IgG4 is seldom noted with that platform. In contrast, positive signal within lymphocytes was frequently seen on the in situ hybridization platform. This phenomenon was observed particularly in cases for which the tissue had been obtained within three years. RNA degradation over time may diminish the likelihood of positive lymphocyte reactivity among archived samples but this should not be an issue for freshly obtained samples. Strong lymphocyte reactivity with the IgG stain was observed in both the IgG4-related disease cases and in patients whose conditions mimicked this disorder. This was not surprising, given the larger number of probes used for the IgG stain. The IgG4 probe target region, however, spans a smaller sequence of nucleotides and therefore accommodates a smaller number of probes, thereby leading to a relatively weaker signal in comparison to IgG. Despite this, in the IgG4-related disease mimickers cohort, only occasional lymphocytes were positive for IgG4 by in situ hybridization. These were primarily cases that are known to show large numbers of IgG4-positive plasma cells in some occasions, such as granulomatosis with polyangiitis (formerly Wegener's) and rheumatoid arthritis.¹¹ Patients with either of these distinct clinical entities often share the property of having an elevated concentration of IgG4 in either their blood or tissues.^{6, 12} In 4 cases of IgG4-related disease, sheets of IgG4-positive lymphocytes were detected. This finding was not observed in any of the control samples. Although these preliminary results suggest that the IgG4 in situ hybridization signal within lymphocytes may serve as a diagnostic marker for IgG4 related disease, additional studies are needed to validate this possibility. The 2 diseases in which IgG4 in situ hybridization signals were detected in small numbers of lymphocytes, granulomatosis with polyangiitis and rheumatoid arthritis, are generally easily distinguished from IgG4 related disease on clinical and serological grounds.

The presence of IgG4 mRNA within lymphocytes confirms the occurrence of isotype switching in these cells. Moreover, this finding suggests that these are post-germinal center cells and that they therefore represent either plasmablasts or memory B-cells.¹³ This observation is compatible with the emerging understanding of the impact and mechanism of B cell depletion strategies in the treatment of IgG4-RD.^{13,14,15} Patients with IgG4-RD demonstrate a swift, targeted response to treatment with rituximab, which binds the CD20 antigen and leads to the depletion of peripheral blood B lymphocytes within approximately two weeks.^{13,14,15} Following rituximab therapy, serum IgG4 concentrations decline precipitously while the concentrations of IgG1, IgG2, IgG3, IgM, and IgA generally remain stable.^{13,14,15} It is possible that these IgG4-bearing lymphocytes identified by in situ hybridization, whose numbers may exceed those of IgG4-bearing plasma cells, are short-lived memory B cells. Their depletion, by virtue of their positivity for the CD20 marker, leads directly to the failure of repletion of short-lived plasma cells, which are the likely source of the serum IgG4 hypergammaglobulinemia observed so often in this context. These IgG4 positive lymphocytes may also play a pivotal role in maintaining the expansion of Th2 effector or effector memory cells, perhaps by promoting antigen presentation.¹⁶ B cells are required for the maintenance of CD4+ memory T cells and may provide specialized antigen-presenting capacity in addition to dendritic cells.¹⁷ It is worth noting that granulomatosis with polyangiitis, another disease that often demonstrates elevated concentrations of IgG4-positive lymphocytes within tissue, also responds readily to rituximab.¹²

### REFERENCES

1. Stone J H, Zen Y, Deshpande V. IgG4-related disease. N Engl J Med 2012; 366(6):539-51.)
2. Sah R P, Chari S T, Pannala R, et al. Differences in clinical profile and relapse rate of type 1 versus type 2 autoimmune pancreatitis. Gastroenterology 2010; 139(1):140-8; quiz e12-3.
3. Ghazale A, Chari S T, Zhang L, et al. Immunoglobulin G4-associated cholangitis: clinical profile and response to therapy. Gastroenterology 2008; 134(3):706-15.
4. Deshpande V. The pathology of IgG4-related disease: critical issues and challenges. Semin Diagn Pathol 2012; 29(4):191-6.
5. Deshpande V, Zen Y, Chan J K, et al. Consensus statement on the pathology of IgG4-related disease. Mod Pathol 2012.
6. Strehl J D, Hartmann A, Agaimy A. Numerous IgG4-positive plasma cells are ubiquitous in diverse localised non-specific chronic inflammatory conditions and need to be distinguished from IgG4-related systemic disorders. J Clin Pathol 2011; 64(3):237-43.
7. Deshpande V, Mino-Kenudson M, Brugge W, Lauwers G Y. Autoimmune pancreatitis: more than just a pancreatic disease? A contemporary review of its pathology. Arch Pathol Lab Med 2005; 129(9):1148-54.
8. Kamisawa T, Funata N, Hayashi Y, et al. A new clinicopathological entity of IgG4-related autoimmune disease. J Gastroenterol 2003; 38(10):982-4.
9. Deshpande V, Khosroshahi A, Nielsen G P, Hamilos D L, Stone J H. Eosinophilic angiocentric fibrosis is a form of IgG4-related systemic disease. Am J Surg Pathol 2011; 35(5):701-6.
10. Deshpande V, Gupta R, Sainani N, et al. Subclassification of autoimmune pancreatitis: a histologic classification with clinical significance. Am J Surg Pathol 2011; 35(1):26-35.
11. Chang S Y, Keogh K, Lewis J E, Ryu J H, Yi E S. Increased IgG4-Positive Plasma Cells in Granulomatosis with Polyangiitis: A Diagnostic Pitfall of IgG4-Related Disease. International journal of rheumatology 2012; 2012:121702.
12. Stone J H, Merkel P A, Spiera R, et al. Rituximab versus cyclophosphamide for ANCA-associated vasculitis. N Engl J Med 2010; 363(3):221-32.
13. Khosroshahi A, Bloch D B, Deshpande V, Stone J H. Rituximab therapy leads to rapid decline of serum IgG4 levels and prompt clinical improvement in IgG4-related systemic disease. Arthritis Rheum 2010; 62(6):1755-62.
14. Khosroshahi A, Stone J H. Treatment approaches to IgG4-related systemic disease. Curr Opin Rheumatol 2011; 23(1):67-71.
15. Khosroshahi A, Carruthers M N, Deshpande V, et al. Rituximab for the treatment of IgG4-related disease: lessons from 10 consecutive patients. Medicine (Baltimore) 2012; 91(1):57-66.
16. Cheuk W, Chan J K. Lymphadenopathy of IgG4-related disease: an underdiagnosed and overdiagnosed entity. Semin Diagn Pathol 2012; 29(4):226-34.
17. Sweetser S, Ahlquist D A, Osborn N K, et al. Clinicopathologic features and treatment outcomes in Cronkhite-Canada syndrome: support for autoimmunity. Dig Dis Sci 2012; 57(2):496-502.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of diagnosing a tumefactive lesion associated with an IgG4-related disease (IgG4-RD) in a subject who has a mass, the method comprising:
contacting a sample comprising plasma cells from the mass with one or more polynucleotide probes that bind specifically to IgG4 mRNA in situ, and one or more polynucleotide probes that bind specifically to IgG mRNA in situ;
detecting binding of the probes to IgG4 mRNA and IgG mRNA in plasma cells in the sample, to determine numbers of IgG4-plasma cells and IgG-plasma cells;
calculating a ratio of IgG4-plasma cells to IgG-plasma cells;
and
identifying a sample in which the ratio of IgG4-plasma cells to IgG-plasma cells is above a threshold as a tumefactive lesion associated with an IgG4-RD, or
identifying a sample in which the IgG4-plasma cells to IgG-plasma cells ratio is below a threshold as not being a tumefactive lesion associated with an IgG4-RD.

2. A method of selecting a treatment for a subject who has a mass, the method comprising:
contacting a sample comprising plasma cells from the mass with one or more polynucleotide probes that bind specifically to IgG4 mRNA in situ, and one or more polynucleotide probes that bind specifically to IgG mRNA in situ;
detecting binding of the probes to IgG4 mRNA and IgG mRNA in plasma cells in the sample, to determine numbers of IgG4-plasma cells and IgG-plasma cells;
calculating a ratio of IgG4-plasma cells to IgG-plasma cells;
and
identifying a sample in which the ratio of IgG4-plasma cells to IgG-plasma cells is above a threshold as a tumefactive lesion associated with an IgG4-RD, and selecting for the subject a treatment for an IgG4-RD; or
identifying a sample in which the IgG4-plasma cells to IgG-plasma cells ratio is below a threshold as not being a tumefactive lesion associated with an IgG4-RD.

3. A method of treating a subject who has a mass, the method comprising:
contacting a sample comprising plasma cells from the mass with one or more polynucleotide probes that bind specifically to IgG4 mRNA in situ, and one or more polynucleotide probes that bind specifically to IgG mRNA in situ;
detecting binding of the probes to IgG4 mRNA and IgG mRNA in plasma cells in the sample, to determine numbers of IgG4-plasma cells and IgG-plasma cells;
calculating a ratio of IgG4-plasma cells to IgG-plasma cells;
and
identifying a sample in which the ratio of IgG4-plasma cells to IgG-plasma cells is above a threshold as a tumefactive lesion associated with an IgG4-RD, and administering to the subject a treatment for an IgG4-RD; or
identifying a sample in which the IgG4-plasma cells to IgG-plasma cells ratio is below a threshold as not being a tumefactive lesion associated with an IgG4-RD.

4. A method of making a differential diagnosis between a mass that is a tumefactive lesion associated with an IgG4-RD or a mass that is not a tumefactive lesion associated with an IgG4-RD in a subject who has a mass, the method comprising:
contacting a sample comprising plasma cells from the mass with one or more polynucleotide probes that bind specifically to IgG4 mRNA in situ, and one or more polynucleotide probes that bind specifically to IgG mRNA in situ;
detecting binding of the probes to IgG4 mRNA and IgG mRNA in plasma cells in the sample, to determine numbers of IgG4-plasma cells and IgG-plasma cells;
calculating a ratio of IgG4-plasma cells to IgG-plasma cells;
and
diagnosing a subject who has a mass in which the ratio of IgG4-plasma cells to IgG-plasma cells is above a threshold as having a tumefactive lesion associated with an IgG4-RD, or diagnosing a subject with a mass in which the ratio of IgG4-plasma cells to IgG-plasma cells is below a threshold as having a tumefactive lesion not associated with an IgG4-RD.

5. The method of claim 1, further comprising identifying a mass that is not a tumefactive lesion associated with an IgG4-RD as being a neoplastic tumor; optionally determining the tissue of origin of the tumor; and optionally selecting and/or administering to the subject a treatment for cancer.

6. The method of claim 1, further comprising determining whether the IgG4-RD is Autoimmune pancreatitis; Eosinophilic angiocentric fibrosis; Fibrosing mediastinitis; Hypertrophic pachymeningitis; Idiopathic hypocomplementemic tubulointerstitialnephritis with extensive tubulointerstitial deposits; Inflammatory aortic aneurysm; Inflammatory pseudotumor; Küttner's tumor (chronic sclerosing sialadenitis); Mediastinal fibrosis; Mikulicz's syndrome; Multifocal fibrosclerosis; Periaortitis and periarteritis; Retroperitoneal fibrosis (Ormond's disease); Riedel's thyroiditis; Sclerosing mesenteritis; Sclerosing pancreatitis; or Sclerosing cholangitis.

7. The method of claim 1, wherein the sample is a biopsy sample obtained from the subject, and preferably wherein the sample comprises a plurality of individually identifiable cells.

8. The method of claim 7, wherein the sample has been fixed, preferably with formalin, optionally embedded in a matrix, and wherein the sample has been sliced into sections.

9. The method of claim 8, wherein:
(i) the one or more polynucleotide probes that bind specifically to IgG4 mRNA in situ, and the one or more polynucleotide probes that bind specifically to IgG mRNA in situ, are both applied to a single section from the sample, or
(ii) the one or more polynucleotide probes that bind specifically to IgG4 mRNA in situ, and the one or more polynucleotide probes that bind specifically to IgG mRNA in situ, are applied to consecutive sections from the sample.

10. The method of claim 9, wherein:
the one or more polynucleotide probes that bind specifically to IgG4 mRNA in situ, and the one or more polynucleotide probes that bind specifically to IgG mRNA in situ, are both applied to a single section from the sample, and binding of the one or more polynucleotide probes to IgG4 is detected using a first detectable signal, and binding of the one or more polynucleotide probes to IgG is detected using a second detectable signal.

11. The method of claim 1, wherein binding of the probes to IgG4 mRNA and IgG mRNA is detected using imaging, and preferably wherein at least three high power fields (HPF) in the mass are analyzed to determine the number of IgG4-positive and IgG-positive cells.

12. The method of claim 1, comprising detecting binding of the probes to IgG4 mRNA and IgG mRNA in the cytoplasm of the plasma cells in the sample, to determine numbers of IgG4-plasma cells and IgG-plasma cells.

13. The method of claim 1, further comprising detecting levels of IgG4 in serum, wherein the presence of elevated IgG4 in serum, plus the presence of the ratio of IgG4-plasma cells to IgG-plasma cells that is above a threshold, indicates that the subject has a tumefactive lesion associated with an IgG4-RD.

14. The method of claim 1, further comprising evaluating the morphology of the cells in the sample, and
(i) identifying a sample having abundant inflammatory cells, mainly plasma cells, fibrosis and obliterative phlebitis, and a ratio of IgG4-plasma cells to IgG-plasma cells is above a threshold as being from a early- or mid-stage tumefactive lesion associated with an IgG4-RD;
(ii) identifying a sample having extensive fibrosis with few plasma cell inflammatory infiltrates and ratio of IgG4-plasma cells to IgG-plasma cells is above a threshold as being from an advanced tumefactive lesion associated with an IgG4-RD; or
(iii) identifying a sample having abundant inflammatory cells, mainly plasma cells, and fibrosis, and ratio of IgG4-plasma cells to IgG-plasma cells below a threshold, as being from a neoplastic tumor.

15. The method of claim 1, comprising:
identifying a sample in which the ratio of IgG4-plasma cells to IgG-plasma cells is above a threshold;
detecting IgKC and IgLC mRNA in the cells in the sample; and
identifying a sample that has IgKC/IgLC clonality as being a IgG4 related lymphoma, or
identifying a sample that does not have IgK/IgL clonality as being a tumefactive lesion associated with an IgG4-RD.

16. The method of claim 1, wherein the one or more probes comprise probes that bind to a plurality of target regions in the IgG4 or IgG mRNA.

17. The method of claim 1, wherein:
the one or more probes that bind to IgG4 mRNA bind to a non-homologous constant region of Homo sapiens Ig heavy chain gamma4, optionally within the sequence CAGCTTGGGCACGAAGACCTACACCTGCAACGTAGATCACAAGCCCAG CAACACCAAGGTGGACAAGAGAGTTGAGTCCAAATATGGTCCCCCATG CCCATCATGCCCAGCACCTGAGTTCCTGGGGGACCATCAGTCTTCCTGT TCCCCCCAAAACC (SEQ ID NO:1); and/or
the one or more probes that bind to IgG mRNA bind to a conserved constant region of the four Ig heavy gamma sequences, optionally within the double-underlined portions of the following sequence:
(SEQ ID NO: 2)
GCAAGCTTCAAGGGCCCATCGGTCTTCCCCCTGGTGCCCTGCTCCAGGAG
CACCTCCGAGAGCACAGCCGCCCTGGGCTGCCTGGTCAAGGACTACTTCC
CCGAACCGGTGACGGTGTCGTGGAACTCATGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACGAAGACCTACACCTGCA
ACGTAGATCACAAGCCCAGCAACACCAAGGTGGACAAGAGAGTTGAGTCC
AAATATGGTCCCCCATGCCCATCATGCCCAGCACCTGAGTTCCTGGGGGG
ACCATCAGTCTTCCTGTTCCCCCCAAAACCCAAGGACACTCTCATGATCT
CCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCA
GGGTCCTCACCGTCCTGCACCAGGACTGGCTGAACGGTAAGGAGTACAAG
TGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCAT
CCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCC
GGAGGACAACTACAAGACCACGCCTCCCGTGCTGGACTCCGACGGCTCCT
TCTTCCTCTACAGCAGGCTAACCGTGGACAAGAGCAGGTGGCAGGAGGGG
AATGTCTTCTCATGCTCCGTGATGCATGAGGCTCTGCACAACCACTACAC
ACAGAAGAGCCTCTCCCTGTCTCCGGGTAAA.

18. The method of claim 17, wherein:
the one or more probes that bind to IgG4 mRNA comprises probes that hybridize to at least 2, 3, 4, 5, 6, 7, or 8 different target sequences within the non-homologous constant region of Homo sapiens Ig heavy chain gamma4, optionally within the sequence CAGCTTGGGCACGAAGACCTACACCTGCAACGTAGATCACAAGCCCAG CAACACCAAGGTGGACAAGAGAGTTGAGTCCAAATATGGTCCCCCATG CCCATCATGCCCAGCACCTGAGTTCCTGGGGGACCATCAGTCTTCCTGT TCCCCCCAAAACC (SEQ ID NO:1); and/or
the one or more probes that bind to IgG mRNA comprises probes that hybridize to at least 2, 3, 4, 5, 6, 7, 8, 9, 10, 11, 12, 13, 14, 15, 16, 17, 18, 19, 20, 21, 22, 23, 24, 25, or 26 different target sequences within the bind to a conserved constant region of the four Ig heavy gamma sequences, optionally within the double-underlined portions of the following sequence:
(SEQ ID NO: 2)
GCAAGCTTCAAGGGCCCATCGGTCTTCCCCCTGGTGCCCTGCTCCAGGAG
CACCTCCGAGAGCACAGCCGCCCTGGGCTGCCTGGTCAAGGACTACTTCC
CCGAACCGGTGACGGTGTCGTGGAACTCATGCGCCCTGACCAGCGGCGTG
CACACCTTCCCGGCTGTCCTACAGTCCTCAGGACTCTACTCCCTCAGCAG
CGTGGTGACCGTGCCCTCCAGCAGCTTGGGCACGAAGACCTACACCTGCA
ACGTAGATCACAAGCCCAGCAACACCAAGGTGGACAAGAGAGTTGAGTCC
AAATATGGTCCCCCATGCCCATCATGCCCAGCACCTGAGTTCCTGGGGGG
ACCATCAGTCTTCCTGTTCCCCCCAAAACCCAAGGACACTCTCATGATCT
CCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGC
CAAGACAAAGCCGCGGGAGGAGCAGTTCAACAGCACGTACCGTGTGGTCA
GGGTCCTCACCGTCCTGCACCAGGACTGGCTGAACGGTAAGGAGTACAAG
TGCAAGGTCTCCAACAAAGGCCTCCCGTCCTCCATCGAGAAAACCATCTC
CAAAGCCAAAGGGCAGCCCCGAGAGCCACAGGTGTACACCCTGCCCCCAT
CCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCC
GGAGGACAACTACAAGACCACGCCTCCCGTGCTGGACTCCGACGGCTCCT
TCTTCCTCTACAGCAGGCTAACCGTGGACAAGAGCAGGTGGCAGGAGGGG
AATGTCTTCTCATGCTCCGTGATGCATGAGGCTCTGCACAACCACTACAC
ACAGAAGAGCCTCTCCCTGTCTCCGGGTAAA.

19. The method of claim 1, wherein the binding of the probes to IgG4 mRNA and IgG mRNA is detected using one or more labels that are directly or indirectly bound to the polynucleotide probes.

20. The method of claim 1, wherein the binding of the probes to IgG4 mRNA is detected using branched nucleic acid signal amplification.

21. The method of claim 20, wherein the probes are branched DNA probes.

22. The method of claim 21, comprising contacting the sample with a plurality of probes that comprises one or more label extender probes that bind to one or more target regions in the IgG4 mRNA; hybridizing one or more pre-amplifier probes to the one or more label extender probes; hybridizing one or more amplifier probes to the pre-amplifier probes; and hybridizing one or more label probes to the one or more amplifier probes.

23. The method of claim 21, comprising contacting the sample with a plurality of probes that comprises one or more label extender probes that bind to one or more target regions in the IgG mRNA; hybridizing one or more pre-amplifier probes to the one or more label extender probes; hybridizing one or more amplifier probes to the pre-amplifier probes; and hybridizing one or more label probes to the one or more amplifier probes.

24. The method of claim 22, wherein the label probes are conjugated to an enzyme, and binding of the probe is detected using a chromogen substrate with the enzyme.

25. The method of claim 22, wherein the label probes are conjugated to a fluorophore, and binding of the probe is detected by observation of emissions from the fluorophore after illumination suitable to excite the fluorophore.

26. The method of claim 1, further comprising:
contacting a sample comprising tissue from the tumor with one or more polynucleotide probes that bind specifically to mRNA encoding a housekeeping gene (HKG) in situ;
detecting binding of the one or more probes to HKG mRNA, and
selecting for further analysis a sample in which binding of the one or more probes to the HKG mRNA is detected, or rejecting a sample in which binding of the one or more probes to the HKG mRNA is not detected.

27. The method of claim 26, wherein the binding of the probes to IgG4 mRNA, IgG mRNA, and/or HKG mRNA is detected using branched nucleic acid signal amplification.

28. The method of claim 27, wherein the probes are branched DNA probes.

29. The method of claim 28, comprising contacting the sample with a plurality of probes that comprises one or more label extender probes that bind to a plurality of target regions in the IgG4, IgG, and/or HKG mRNA; hybridizing one or more pre-amplifier probes to the one or more label extender probes; hybridizing one or more amplifier probes to the pre-amplifier; and hybridizing one or more label probes to the one or more amplifier probes.

30. The method of claim 29, wherein the one or more polynucleotide probes that bind specifically to IgG4 mRNA in situ and the one or more polynucleotide probes that bind specifically to IgG mRNA in situ are applied to consecutive sections from the sample, the label probes are conjugated to an enzyme, binding of the IgG4 probes to IgG4 mRNA and IgG probes to IgG mRNA is detected using a first chromogen substrate for the enzyme, and binding of the HKG probes to HKG mRNA is detected using a second chromogen substrate for the enzyme.

31. The method of claim 29, wherein the one or more polynucleotide probes that bind specifically to IgG4 mRNA in situ and the one or more polynucleotide probes that bind specifically to IgG mRNA in situ are applied to consecutive sections from the sample, the label probes are conjugated to a fluorophore, binding of the IgG4 probes to IgG4 mRNA and IgG probes to IgG mRNA is detected using a first fluorophore, and binding of the HKG probes to HKG mRNA is detected using a second fluorophore.

32. The method of claim 29, wherein the one or more polynucleotide probes that bind specifically to IgG4 mRNA in situ and the one or more polynucleotide probes that bind specifically to IgG mRNA in situ are both applied to a single section from the sample, the label probes are conjugated to an enzyme, binding of the IgG4 probes to IgG4 mRNA is detected using a first chromogen substrate for the enzyme, IgG probes to IgG mRNA is detected using a second chromogen substrate for the enzyme, and binding of the HKG probes to HKG mRNA is detected using a third chromogen substrate for the enzyme.

33. The method of claim 29, wherein the one or more polynucleotide probes that bind specifically to IgG4 mRNA in situ and the one or more polynucleotide probes that bind specifically to IgG mRNA in situ are both applied to a single section from the sample, the label probes are conjugated to a fluorophore, binding of the IgG4 probes to IgG4 mRNA is detected using a first fluorophore, binding of the IgG probes to IgG mRNA is detected using a second fluorophore, and binding of the HKG probes to HKG mRNA is detected using a third fluorophore.

34. A kit for performing the method of claim 1, wherein the kit comprises:
i. one or more polynucleotide probes that bind specifically to IgG4 mRNA in situ comprising one or more label extender probes that are capable of binding to one or more target regions in the IgG4 mRNA; and
ii. one or more polynucleotide probes that bind specifically to IgG mRNA in situ.

35. The kit of claim 34, wherein the one or more polynucleotide probes that bind specifically to IgG mRNA in situ comprise one or more label extender probes that are capable of binding to one or more target regions in the IgG mRNA.

36. The kit of claim 34, wherein the kit further comprises one or more polynucleotide probes that bind specifically to IgKC mRNA in situ and one or more polynucleotide probes that bind specifically to IgLC mRNA in situ.

37. The kit of any claim 34, wherein the kit further comprises one or more polynucleotide probes that bind specifically to mRNA encoding a housekeeping gene (HKG) in situ.
